(19) United States
(12) Patent Application Publication
(10) Pub. No.: US 2019/0292605 A1
(43) Pub. Date: Sep. 26, 2019

(54) Biomarkers of Response to Cyclin D-CDK4/6 Targeted Therapies in Cancer

(71) Applicant: The Regents of the University of California, Oakland, CA (US)

(72) Inventors: Dennis Slamon, Woodland Hills, CA (US); Richard Finn, Encino, CA (US); Dylan Conklin, Woodland Hills, CA (US); Erika von Euw, Los Angeles, CA (US)

(21) Appl. No.: 16/369,963

(22) Filed: Mar. 29, 2019

Related U.S. Application Data
(63) Continuation of application No. 15/123,351, filed on Sep. 2, 2016, now abandoned.
(60) Provisional application No. 61/947,667, filed on Mar. 4, 2014.

(51) Int. Cl. C12Q 1/6886; A61K 39/395; A61K 31/519; C07K 16/32; A61K 45/06

(52) U.S. Cl. CPC C12Q 1/6886; A61K 39/39558; A61K 31/519; C07K 16/32; C07K 2317/24; C12Q 2600/106; C12Q 2600/158; C12Q 2600/156; A61K 45/06

(57) ABSTRACT

Provided herein are biomarkers indicating sensitivity or resistance to Cyclin D-CDK4/6 inhibitor therapy in cancer and methods of use.

### RELATED APPLICATIONS AND INCORPORATION BY REFERENCE

This application is a Continuation of U.S. application Ser. No. 15/123,351, filed on Sep. 2, 2016, which is a national stage filing under 35 U.S.C. § 371 of PCT Application No: PCT/US15/18831, filed on Mar. 4, 2015, which claims the benefit of priority of U.S. Provisional Application No. 61/947,667, filed on Mar. 4, 2014. The entire contents of these applications are hereby incorporated by reference in their entirety.

### FIELD OF THE DISCLOSURE

The invention relates generally to biomarkers indicating response to CYCLIN D-CDK4/6 inhibitor therapy, and more specifically to genetic markers indicating sensitivity or resistance to treatment with palbociclib in cancer.

### BACKGROUND OF THE DISCLOSURE

Several cyclin dependent kinase (CDK) inhibitors have been evaluated in cancer medicine without significant clinical activity. For example, palbociclib is a highly selective inhibitor of cyclin dependent kinases 4 and 6 (CDK4/6) that has been shown to inhibit growth of malignant cell lines in vitro and in vivo by preventing the phosphorylation of Rb and stopping the progression G1/S of cell cycle.

Clinical trials are currently underway with palbociclib as well as other CDK4/6 inhibitors and most of these trials have been designed as “biomarker-independent” in that there is no biomarker selection for trial inclusion. As a result, these trials may show little or no efficacy compared to those trials enriched for patients with genomic biomarkers of susceptibility to CDK4/6 inhibition. Thus, a need exists for validated biomarkers for identifying patients who may be uniquely sensitive to treatment with CDK4/6 inhibitors across cancers of differing histologies.

### SUMMARY OF THE DISCLOSURE

The present invention relates to biomarkers indicating sensitivity or resistance to a CDK4 inhibitor, a CDK6 inhibitor, or a cyclin D inhibitor and methods of use. Accordingly there are provided methods of identifying a subject having a CDK4 inhibitor-, CDK6 inhibitor-, or cyclin D inhibitor-sensitive tumor, by detecting a loss-of-function point mutation in CDH1, chromosomal amplification of 17q12-21 (ERBB2), chromosomal amplification of 11q13 (CCND1), chromosomal amplification of 1q (MDM4), or combinations thereof in a tumor sample from the subject, wherein detection of the loss-of function point mutation or chromosomal amplification is indicative of a tumor that is sensitive to a CDK4 inhibitor, a CDK6 inhibitor, or a cyclin D inhibitor.

Also provided are methods of treating a CDK4 inhibitor-, CDK6 inhibitor-, or cyclin D inhibitor-sensitive tumor in a subject by detecting a loss-of-function point mutation in CDH1, chromosomal amplification of 17q12-21 (ERBB2), chromosomal amplification of 11q13 (CCND1), chromosomal amplification of 1q (MDM4), or combinations thereof in a tumor sample from the subject, wherein detection of the loss-of function point mutation or chromosomal amplification is indicative of a tumor that is sensitive to a CDK4 inhibitor, a CDK6 inhibitor, or a cyclin D inhibitor, and administering an effective amount of a CDK4 inhibitor, a CDK6 inhibitor, or a cyclin D inhibitor to the subject, thereby treating the tumor.

Further provided are methods of treating a tumor with a loss-of-function point mutation in CDH1, chromosomal amplification of 17q12-21 (ERBB2), chromosomal amplification of 11q13 (CCND1), chromosomal amplification of 1q (MDM4), or combinations thereof in a subject by administering an effective amount of a CDK4 inhibitor, a CDK6 inhibitor, or a cyclin D inhibitor to a subject having a tumor with a loss-of-function point mutation in CDH1, chromosomal amplification of 17q12-21 (ERBB2), chromosomal amplification of 11q13 (CCND1), chromosomal amplification of 1q (MDM4), or combinations thereof, thereby treating the tumor.

Also provided are methods of identifying a subject having a CDK4 inhibitor-, CDK6 inhibitor-, or cyclin D inhibitor-resistant tumor, by detecting an activating point mutation of the smoothened (SMO) gene, an activating point mutation of the RET proto-oncogene, a chromosomal amplification of 19q12 (CCNE1), a loss-of-function point mutation of FBXW7, a loss-of-function point mutation of retinoblastoma (RB1), a chromosomal deletion of RB1, a chromosomal amplification of sonic hedgehog (SHH), high baseline GLI2 mRNA expression, high baseline SMO mRNA expression, or combinations thereof in a tumor sample from the subject, wherein detection of any of the foregoing point mutations, chromosomal deletions, high baseline mRNA expression, or chromosomal amplifications is indicative of a tumor that is resistant to a CDK4 inhibitor, a CDK6 inhibitor, or a cyclin D inhibitor.

Also provided are methods of identifying a subject having a CDK4 inhibitor-, CDK6 inhibitor-, or cyclin D inhibitor-sensitive tumor, by detecting a cyclin D1 amplification, a HER2 amplification, or combinations thereof in a tumor sample from the subject, wherein detection of the amplification is indicative of a tumor that is sensitive to a CDK4 inhibitor, a CDK6 inhibitor, or a cyclin D inhibitor.

Also provided are methods of identifying a subject having a CDK4 inhibitor-, CDK6 inhibitor-, or cyclin D inhibitor-resistant tumor, by detecting a cyclin E amplification in a tumor sample from the subject, wherein detection of the amplification is indicative of a tumor that is resistant to a CDK4 inhibitor, a CDK6 inhibitor, or a cyclin D inhibitor.

Further provided are methods of identifying a subject having a CDK4 inhibitor-, CDK6 inhibitor-, or cyclin D inhibitor-resistant tumor, by detecting a p16 loss or a p21 gain in a tumor sample from the subject, wherein detection of the p16 loss or the p21 gain is indicative of a tumor that is resistant to a CDK4 inhibitor, a CDK6 inhibitor, or a cyclin D inhibitor.

In various embodiments, the disclosure provides a method for treating a subject having cancer or tumor that is resistant to a CDK4 inhibitor, CDK6 inhibitor, or cyclin D inhibitor comprising identifying a subject having a CDK4 inhibitor-, CDK6 inhibitor-, or cyclin D inhibitor-resistant tumor and administering a therapeutic agent that is not a CDK4 inhibitor-, CDK6 inhibitor-, or cyclin D inhibitor. In various embodiments, the CDK4 inhibitor-, CDK6 inhibitor-, or cyclin D inhibitor-resistant tumor comprises one or more of the following mutations selected from the group consisting of an activating point mutation of the smoothened (SMO) gene, an activating point mutation of the RET proto-oncogene, a chromosomal amplification of 19q12 (CCNE1), a loss-of-function point mutation of FBXW7, a loss-of-function point mutation of retinoblastoma (RB1), a chromosomal deletion of RB1, a chromosomal amplification of sonic hedgehog (SHH), high baseline GLI2 mRNA expression, and high baseline SMO mRNA expression.

In various embodiments, the disclosure provides a method for treating a subject having cancer that is sensitive to a CDK4 inhibitor, CDK6 inhibitor, or cyclin D inhibitor comprising identifying a subject having a CDK4 inhibitor-, CDK6 inhibitor-, or cyclin D inhibitor-resistant tumor and administering an effective amount of a CDK4 inhibitor-, CDK6 inhibitor-, or cyclin D inhibitor. In various embodiments, the CDK4 inhibitor-, CDK6 inhibitor-, or cyclin D inhibitor-sensitive cancer comprises a loss-of-function point mutation in CDH1, chromosomal amplification of 17q12-21 (ERBB2), chromosomal amplification of 11q13 (CCND1), chromosomal amplification of 1q (MDM4), a cyclin D1 amplification, a HER2 amplification or combinations thereof.

The disclosure also contemplates a method of predicting whether a cancer cell or tumor is sensitive to treatment with a CDK4 inhibitor, CDK6 inhibitor, or cyclin D inhibitor comprising obtaining a sample of a tumor or cancer cell, and measuring genome mutations, genome copy number, protein expression levels and mRNA expression levels, wherein when the cancer cell or tumor expresses one or more of the following criteria: a loss-of-function point mutation in CDH1, chromosomal amplification of 17q12-21 (ERBB2), chromosomal amplification of 11q13 (CCND1), chromosomal amplification of 1q (MDM4), a cyclin D1 amplification, a HER2 amplification or combinations thereof, it is indicative of a cancer cell or tumor is sensitive to a CDK4 inhibitor, CDK6 inhibitor, or cyclin D inhibitor.

In various embodiments, the disclosure provides a method of predicting whether a cancer cell or tumor is resistant to treatment with a CDK4 inhibitor, CDK6 inhibitor, or cyclin D inhibitor comprising obtaining a sample of a tumor or cancer cell, and measuring genome mutations, genome copy number, protein expression levels and mRNA expression levels, wherein when the cancer cell or tumor expresses one or more of the following criteria: an activating point mutation of the smoothened (SMO) gene, an activating point mutation of the RET proto-oncogene, a chromosomal amplification of 19q12 (CCNE1), a loss-of-function point mutation of FBXW7, a loss-of-function point mutation of retinoblastoma (RB1), a chromosomal deletion of RB1, a chromosomal amplification of sonic hedgehog (SHH), high baseline GLI2 mRNA expression, high baseline SMO mRNA expression or combinations thereof, it is indicative of a cancer cell or tumor is resistant to a CDK4 inhibitor, CDK6 inhibitor, or cyclin D inhibitor.

Also contemplated is a method of diagnosing whether a subject has a CDK4 inhibitor-, CDK6 inhibitor-, or cyclin D inhibitor-sensitive cancer, comprising: a) obtaining samples from the subject to provide a set of target polynucleotides and proteins; b) contacting the target polynucleotides or proteins to a microarray to provide a gene and protein expression profile for the test sample; and c) comparing the test sample expression profile to a profile generated from a control sample, wherein a CDK4 inhibitor-, CDK6 inhibitor-, or cyclin D inhibitor-sensitive cancer comprises i) a loss-of-function point mutation in CDH1; ii) chromosomal amplification of 17q12-21 (ERBB2); iii) chromosomal amplification of 11q13 (CCND1); iv) chromosomal amplification of 1q (MDM4); v) a cyclin D1 amplification; vi) a HER2 amplification; or vii) combinations of i) to iv).

Also provided is a method of diagnosing whether a subject has a CDK4 inhibitor-, CDK6 inhibitor-, or cyclin D inhibitor-resistant cancer, comprising: a) obtaining samples from the subject to provide a set of target polynucleotides and proteins; b) contacting the target polynucleotides or proteins to a microarray to provide a gene and protein expression profile for the test sample; and c) comparing the test sample expression profile to a profile generated from a control sample, wherein a CDK4 inhibitor-, CDK6 inhibitor-, or cyclin D inhibitor-resistant cancer comprises i) an activating point mutation of the smoothened (SMO) gene; ii) an activating point mutation of the RET proto-oncogene; iii) a chromosomal amplification of 19q12 (CCNE1); iv) a loss-of-function point mutation of FBXW7; v) a loss-of-function point mutation of retinoblastoma (RB1); vi) a chromosomal deletion of RB1; vii) a chromosomal amplification of sonic hedgehog (SHH); viii) high baseline GLI2 mRNA expression; ix) high baseline SMO mRNA expression; or x) combinations of i) to ix).

In various embodiments, the disclosure provides a method of enhancing the effects of a chemotherapeutic agent in the treatment of cancer comprising administering a chemotherapeutic agent to a subject in combination with a CDK4 inhibitor, CDK6 inhibitor, or cyclin D inhibitor.

In various embodiments, the cancer is sensitive to treatment with a CDK4 inhibitor, CDK6 inhibitor, or cyclin D inhibitor. In various embodiments, the cancer is gastric cancer and the chemotherapeutic agent is trastuzumab. In various embodiments, the cancer is melanoma and the chemotherapeutic agent is an inhibitor of Smoothened.

In various embodiments, the disclosure contemplates a method for treating a subject having colon cancer that is sensitive to a CDK4 inhibitor, CDK6 inhibitor, or cyclin D inhibitor comprising identifying a subject having a CDK4 inhibitor-, CDK6 inhibitor-, or cyclin D inhibitor-sensitive colon cancer and administering a CDK4 inhibitor, CDK6 inhibitor, or cyclin D inhibitor. In various embodiments, the CDK4 inhibitor-, CDK6 inhibitor-, or cyclin D inhibitor-sensitive colon cancer comprises i); high p16 protein; ii) copy number amplification of RB1; or iii) combinations of i) and ii).

In various embodiments, the CDK4 inhibitor-, CDK6 inhibitor-, or cyclin D inhibitor-sensitive colon cancer does not comprise i) loss-of-function mutations in FBXW7, ii) point mutations in RET; iii) CCNE1 copy number amplifications, iv) or combinations of any of i), ii) or iii). In various embodiments, a CDK4 inhibitor-, CDK6 inhibitor-, or cyclin D inhibitor-resistant colon cancer comprises, i) loss-of-function mutations in FBXW7, ii) point mutations in RET; iii) CCNE1 copy number amplifications, iv) or combinations of any of i), ii) or iii).

In various embodiments, the disclosure contemplates a method for treating a subject having gastric cancer that is sensitive to a CDK4 inhibitor, CDK6 inhibitor, or cyclin D inhibitor comprising identifying a subject having a CDK4 inhibitor-, CDK6 inhibitor-, or cyclin D inhibitor-sensitive gastric cancer and administering a CDK4 inhibitor, CDK6 inhibitor, or cyclin D inhibitor. In various embodiments, the CDK4 inhibitor, CDK6 inhibitor, or cyclin D inhibitor-sensitive gastric cancer comprises i) CCND1 amplification; ii) ERBB2 amplification; or iii) combinations of i) and ii).

In various embodiments, the CDK4 inhibitor-, CDK6 inhibitor-, or cyclin D inhibitor-sensitive gastric cancer does not comprise i) loss-of-function mutations in FBXW7, ii) loss-of-function mutations in RB1; iii) CCNE1 copy number amplification; or iv) combinations of any of i), ii) or iii). In various embodiments, a CDK4 inhibitor-, CDK6 inhibitor-, or cyclin D inhibitor-resistant gastric cancer comprises, i) loss-of-function mutations in FBXW7, ii) loss-of-function mutations in RB1; iii) CCNE1 copy number amplification; or iv) combinations of any of i), ii) or iii).

In various embodiments, the disclosure provides a method for treating a subject having melanoma that is sensitive to a CDK4 inhibitor, CDK6 inhibitor, or cyclin D inhibitor comprising identifying a subject having a CDK4 inhibitor-, CDK6 inhibitor-, or cyclin D inhibitor-sensitive melanoma and administering a CDK4 inhibitor, CDK6 inhibitor, or cyclin D inhibitor. In various embodiments, the CDK4 inhibitor-, CDK6 inhibitor-, or cyclin D inhibitor-sensitive melanoma comprises i) decrease in phospho-Rb1 protein; ii) decrease in FOXM1 protein; or iii) combinations of i) and ii). In various embodiments, the CDK4 inhibitor-, CDK6 inhibitor-, or cyclin D inhibitor-sensitive melanoma does not comprise i) activation of SHH; ii) overexpression of the SMO gene; iii) overexpression of the GL12 gene; or iv) combinations of i), ii) and iii). In various embodiments, a CDK4 inhibitor-, CDK6 inhibitor-, or cyclin D inhibitor-sensitive melanoma comprises i) activation of SHH; ii) overexpression of the SMO gene; iii) overexpression of the GL12 gene; or iv) combinations of i), ii) and iii).

In some embodiments, an effective dosage of inhibitor may be within the range of 0.01 mg to 1000 mg per kg (mg/kg) of body weight per day. In some embodiments, the inhibitor is administered at a daily dose ranging from about 10 mg/kg to about 250 mg/kg, or from about 100 mg/kg to about 250 mg/kg, or from about 60 mg/kg to about 100 mg/kg or from about 50 mg/kg to about 90 mg/kg, or from about 30 mg/kg to about 80 mg/kg, or from about 20 mg/kg to about 60 mg/kg, or from about 10 mg/kg to about 50 mg/kg. Further, the effective amount or dose may be 0.5 mg/kg, 1 mg/kg, 5 mg/kg, 10 mg/kg, 15 mg/kg, 20 mg/kg/25 mg/kg, 30 mg/kg, 35 mg/kg, 40 mg/kg, 45 mg/kg, 50 mg/kg, 55 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, 250 mg/kg, 300 mg/kg, 350 mg/kg, 400 mg/kg, 450 mg/kg, 500 mg/kg, and may increase by 25 mg/kg increments up to 1000 mg/kg, or may range between any two of the foregoing values.

Administration may continue for at least 1 month, 2 months, 3 months, 6 months, 9 months, 1 year, 2 years, or more.

In various embodiments, the methods further comprise administering a second agent.

In various embodiments, the second agent is a chemotherapeutic agent or a radiotherapeutic agent. It is contemplated that the CDK4/6 or cyclin D inhibitor is administered in combination with a chemotherapeutic that is the standard of care for the cancer being treated. Exemplary chemotherapeutic agents and radiotherapeutic agents are set out in greater detail in the Detailed Description and standard of care for a particular cancer type is known in the art.

Exemplary modes of administration contemplated herein include enteral or parenteral routes, including, but not limited to, oral, subcutaneous, intramuscular, intravenous, intraperitoneal, topical, transdermal, or transmucosal administration.

In various embodiments, the subject is a mammal. Exemplary mammals include humans, non-human primates such as chimpanzees, and other apes and monkey species; rabbits, dogs, cats, rodents, such as rats, mice and guinea pigs, cattle, horses, sheep, goats, and swine. In one embodiment, the subject is a human.

It is understood that each feature or embodiment, or combination, described herein is a non-limiting, illustrative example of any of the aspects of the disclosure and, as such, is meant to be combinable with any other feature or embodiment, or combination, described herein. For example, where features are described with language such as “one embodiment”, “some embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the disclosure. Where examples of values falling within ranges are disclosed, any of these examples are contemplated as possible endpoints of a range, any and all numeric values between such endpoints are contemplated, and any and all combinations of upper and lower endpoints are envisioned.

### BRIEF DESCRIPTION OF THE DRAWINGS

FIG. 1 shows the IC50 of palbociclib on different cancer cell types.

FIG. 2 contains two panels, FIG. 2A and FIG. 2B, depicting palbociclib IC50 distribution by CCND1 status (amplification) in all cancer types.

FIG. 3 contains two panels, FIG. 3A and FIG. 3B, depicting palbociclib IC50 distribution by CDH1 status (RLOFPM) in all cancer types.

FIG. 4 contains two panels, FIG. 4A and FIG. 4B, depicting palbociclib IC50 distribution by ERBB2 status (amplification) in all cancer types.

FIG. 5 contains two panels, FIG. 5A and FIG. 5B, depicting palbociclib IC50 distribution by MDM4 status (amplification) in all cancer types.

FIG. 6 contains two panels, FIG. 6A and FIG. 6B, depicting palbociclib IC50 distribution by FBXW7 status (RLOFPM) in all cancer types.

FIG. 7 contains two panels, FIG. 7A and FIG. 7B, depicting palbociclib IC50 distribution by RB1 status (RLOFPM) in all cancer types.

FIG. 8 contains two panels, FIG. 8A and FIG. 8B, depicting palbociclib IC50 distribution by SMO status (DAPM) in all cancer types.

FIG. 9 contains two panels, FIG. 9A and FIG. 9B, depicting palbociclib IC50 distribution by RET status (DAPM) in all cancer types.

FIG. 10 contains two panels, FIG. 10A and FIG. 10B, depicting palbociclib IC50 distribution by CCNE1 status (amplification) in all cancer types.

FIG. 11 contains two panels, FIG. 11A and FIG. 11B, depicting palbociclib IC50 distribution by RB1 status (deletion) in all cancer types.

FIG. 12 contains two panels, FIG. 12A and FIG. 12B, depicting palbociclib IC50 distribution by SHH status (amplification) in all cancer types.

FIG. 13 contains two panels, FIG. 13A and FIG. 13B, depicting palbociclib IC50 distribution by SHH status (amplification) in a melanoma panel.

FIG. 14 illustrates SHH chromosomal amplification in all cancer studies (patient tumors).

FIG. 15 shows GLI2 and SMO high mRNA baseline expression is predicted for resistance to palbociclib in melanoma cell lines.

FIG. 16 illustrates SMO high mRNA baseline expression predicted for resistance to Palbociclib in a cell line panel.

FIG. 17 illustrates that effects on the cell cycle after palbociclib treatment in melanoma cell lines.

FIG. 18 contains three panels, FIG. 18A-FIG. 18C, depicting the results of a GL12 knockdown study in palbociclib resistant cell lines CHL-1 (FIG. 18A) and M233 (FIG. 18B) using siRNA. Cell numbers are in the left panel, % inhibition is in the right panel. FIG. 18C shows the results in Palbociclib sensitive cells M296 (FIG. 18C) as a control.

FIG. 19 contains two panels, FIG. 19A and FIG. 19B, showing the effects of palbociclib treatment on resistance cells in combination with GLI2 siRNA knockdown on cell lines expressing high GLI2 protein levels over two doses of palbociclib, 100 nM (FIG. 19A) and 250 nM (FIG. 19B).

FIG. 20 contains two panels, FIG. 20A and FIG. 20B, showing that trastuzumab and palbociclib are synergistic in HER2 amplified gastric cancer cells.

FIG. 21 contains two panels, FIG. 21A and FIG. 21B, showing that palbociclib induces G1/G0 arrest in resistant (FIG. 21A) and sensitive (FIG. 21B) colon cancer cells.

FIG. 22 contains two panels, FIG. 22A and FIG. 22B, showing that palbociclib induces G1/G0 arrest in resistant (FIG. 22A) and sensitive (FIG. 22B) gastric cancer cells.

### DETAILED DESCRIPTION OF THE DISCLOSURE

The present disclosure relates, in general, to identification of biomarkers in cancer cells that are associated with sensitivity or resistance to treatment with CDK4/6 inhibitors and/or cyclin D inhibitors. The identified biomarkers are useful to classify a subject's cancer or tumor as treatable with an CDK4/6 and/or cyclin D inhibitor, and the patient identified as having an CDK4/6 inhibitor- or cyclin D inhibitor-sensitive tumor can then be treated with a CDK4/6 inhibitor and/or a cyclin D inhibitor. Similarly, a cancer or tumor identified as resistant to CDK4/6 inhibitor and/or cyclin D inhibitor should not be treated with an CDK4/6 inhibitor and the method indicates treatment with alternate chemotherapeutics.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

“About” as used herein when referring to a measurable value such as an amount, a temporal duration, and the like, is meant to encompass variations of ±20% or ±10%, more preferably ±5%, even more preferably ±1% from the specified value, as such variations are appropriate to perform the disclosed methods.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

As used herein “an inhibitor of CDK4/6” and “inhibitor of cyclin D” refer to a compound or composition that inhibits activity of CDK4/6 or cyclin D, respectively, e.g., to phosphorylate a serine or threonine residue on proteins, or inhibits the interaction of CDK4/6 or cyclin D with other proteins that may be in the signal pathway. CDK4 and CDK6 form a complex with Cyclin D to regulate cell cycle progression from G1 to S phase. CDK 4 as been shown to also interact with the following proteins: retinoblastoma (Rb), CDC37, CDKN1B, CDKN2B, CDKN2C, CEBPA, CCND1, CCND3, DBNL, MyoD, P16, PCNA, and SERTAD1. CDK6 has been shown to also interact with Cyclin-dependent kinase 6 was shown to interact with retinoblastoma, CDKN2C, PPM1B, Cyclin D3, Cyclin D1 and PPP2CA. It is contemplated that the inhibitor can inhibit the interaction of of CDK4 or CDK6 with any of the proteins listed above. The inhibitor palbociclib and other inhibitors are described in greater detail in the Detailed Description.

As used herein “sensitive to cyclin dependent kinase 4/6 (CDK4/6) inhibitor” or “CDK4/6 i-sensitive cancer” refers to a cell or cancer that has reduced growth in the presence of a CDK4/6 inhibitor compared to in the absence of such an inhibitor. Similarly “sensitive to cyclin D inhibitor” or “cyclin D i-sensitive cancer” refers to a cell or cancer that has reduced growth in the presence of a cyclin D inhibitor compared to in the absence of such an inhibitor. Sensitivity can refer to a cytotoxic or cytostatic effect of the CDK4/6 or cyclin D inhibitor on the cell. It is contemplated that a sensitive cell line can have a 1.5, 2, 3, 4, 5, 6, 7, 8, 9, 10, 15, 20, 25-fold or more change in growth rate in the presence of a CDK4/6 or cyclin D inhibitor. Sensitivity can also be measured by change in genome sequence or copy number of a gene, increase or reduction in particular protein expression or mRNA expression, or other measurement disclosed herein to be a measure of sensitivity. It is contemplated that a sensitive cell line can have a 1.5, 2, 3, 4, 5, 6, 7, 8, 9, 10, 15, 20, 25-fold or more change in one or more sensitive biomarker parameters in the presence of an CDK4/6 inhibitor

As used herein “resistant to a cyclin dependent kinase 4/6 (CDK4/6) inhibitor” or “CDK4/6 i-resistant cancer” refers to a cell or cancer that has normal (or baseline) growth in the presence of a CDK4/6 inhibitor and is substantially similar as in the absence of such an inhibitor. Similarly “resistant to cyclin D inhibitor” or “cyclin D i-resistant cancer” refers to a cell or cancer that has normal (or baseline) growth in the presence of a cyclin D inhibitor and is substantially similar as in the absence of such an inhibitor. Resistance can be measured by a relative maintenance of cell growth rate in the presence of a CDK4/6 or cyclin D inhibitor, or by a change in genome sequence or copy number of a gene, increase or reduction in particular protein expression or mRNA expression, or other measurement disclosed herein to be a measure of resistance. It is contemplated that a resistant cell line can have a 1.5, 2, 3, 4, 5, 6, 7, 8, 9, 10, 15, 20, 25-fold or more change in one or more resistant biomarker parameters in the presence of a CDK4/6 or cyclin D inhibitor.

As used herein, a “therapeutically effective amount” or “effective amount” refers to that amount of an inhibitor described herein, sufficient to result in amelioration of symptoms, for example, treatment, healing, prevention or amelioration of the relevant medical condition, or an increase in rate of treatment, healing, prevention or amelioration of such conditions, typically providing a statistically significant improvement in the treated patient population. When referencing an individual active ingredient, administered alone, a therapeutically effective amount or dose refers to that ingredient alone. When referring to a combination, a therapeutically effective amount or dose refers to combined amounts of the active ingredients that result in the therapeutic effect, whether administered in combination, including serially or simultaneously. In various embodiments, a therapeutically effective amount of the inhibitor ameliorates symptoms associated with various cancers, including but not limited to, loss of appetite, oral pain, upper abdominal pain, fatigue, abdominal swelling, persistent aches, bone pain, nausea, vomiting, constipation, weight loss, headaches, rectal bleeding, night sweats, digestive discomfort, and painful urination.

“Treatment” refers to prophylactic treatment or therapeutic treatment. In certain embodiments, “treatment” refers to administration of a compound or composition to a subject for therapeutic or prophylactic purposes.

A “therapeutic” treatment is a treatment administered to a subject who exhibits signs or symptoms of pathology for the purpose of diminishing or eliminating those signs or symptoms. The signs or symptoms may be biochemical, cellular, histological, functional or physical, subjective or objective.

A “prophylactic” treatment is a treatment administered to a subject who does not exhibit signs of a disease or exhibits only early signs of the disease, for the purpose of decreasing the risk of developing pathology. The compounds or compositions of the disclosure may be given as a prophylactic treatment to reduce the likelihood of developing a pathology or to minimize the severity of the pathology, if developed.

“Pharmaceutical composition” refers to a composition suitable for pharmaceutical use in a subject animal, including humans and mammals. A pharmaceutical composition comprises a therapeutically effective amount of an inhibitor or other product described herein, optionally another biologically active agent, and optionally a pharmaceutically acceptable excipient, carrier or diluent. In an embodiment, a pharmaceutical composition encompasses a composition comprising the active ingredient(s), and the inert ingredient(s) that make up the carrier, as well as any product that results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the disclosure and a pharmaceutically acceptable excipient, carrier or diluent.

“Pharmaceutically acceptable carrier” refers to any of the standard pharmaceutical carriers, buffers, and the like, such as a phosphate buffered saline solution, 5% aqueous solution of dextrose, and emulsions (e.g., an oil/water or water/oil emulsion). Non-limiting examples of excipients include adjuvants, binders, fillers, diluents, disintegrants, emulsifying agents, wetting agents, lubricants, glidants, sweetening agents, flavoring agents, and coloring agents. Suitable pharmaceutical carriers, excipients and diluents are described in Remington's Pharmaceutical Sciences, 19th Ed. (Mack Publishing Co., Easton, 1995). Preferred pharmaceutical carriers depend upon the intended mode of administration of the active agent. Typical modes of administration include enteral (e.g., oral) or parenteral (e.g., subcutaneous, intramuscular, intravenous or intraperitoneal injection; or topical, transdermal, or transmucosal administration).

As used herein “pharmaceutically acceptable” or “pharmacologically acceptable” salt, ester or other derivative of an active agent comprise, for example, salts, esters or other derivatives refers to a material that is not biologically or otherwise undesirable, i.e., the material may be administered to an individual without causing any undesirable biological effects or without interacting in a deleterious manner with any of the components of the composition in which it is contained or with any components present on or in the body of the individual.

As used herein, the term “unit dosage form” refers to physically discrete units suitable as unitary dosages for human and animal subjects, each unit containing a predetermined quantity of a compound of the disclosure calculated in an amount sufficient to produce the desired effect, optionally in association with a pharmaceutically acceptable excipient, diluent, carrier or vehicle. The specifications for the novel unit dosage forms of the present disclosure depend on the particular compound employed and the effect to be achieved, and the pharmacodynamics associated with each compound in the host.

The term “antibody” is meant to include intact molecules of polyclonal or monoclonal antibodies, chimeric, single chain, and humanized antibodies, as well as fragments thereof, such as Fab and F(ab′)2, Fv and SCA fragments which are capable of binding an epitopic determinant. Monoclonal antibodies are made from antigen containing fragments of the protein by methods well known to those skilled in the art (Kohler, et al., Nature, 256:495, 1975). An Fab fragment consists of a monovalent antigen-binding fragment of an antibody molecule, and can be produced by digestion of a whole antibody molecule with the enzyme papain, to yield a fragment consisting of an intact light chain and a portion of a heavy chain. A Fab′ fragment of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner. An (Fab′)2 fragment of an antibody can be obtained by treating a whole antibody molecule with the enzyme pepsin, without subsequent reduction. A (Fab′)2 fragment is a dimer of two Fab′ fragments, held together by two disulfide bonds. An Fv fragment is defined as a genetically engineered fragment containing the variable region of a light chain and the variable region of a heavy chain expressed as two chains. A single chain antibody (“SCA”) is a genetically engineered single chain molecule containing the variable region of a light chain and the variable region of a heavy chain, linked by a suitable, flexible polypeptide linker.

“Nucleic acid” or “oligonucleotide” or “polynucleotide” or grammatical equivalents used herein means at least two nucleotides covalently linked together. Nucleic acids are typically deoxyribonucleotide or ribonucleotides polymers (pure or mixed) in single- or double-stranded form. The term may encompass nucleic acids containing nucleotide analogs or modified backbone residues or linkages, which are synthetic, naturally occurring, and non-naturally occurring, which have similar binding, structural, or functional properties as the reference nucleic acid, and which are metabolized in a manner similar to the reference nucleotides. Non-limiting examples of such analogs include, without limitation, phosphorothioates, phosphoramidates, methyl phosphonates, chiral-methyl phosphonates, 2-0-methyl ribonucleotides, and peptide-nucleic acids (PNAs). A nucleic acid will generally contain phosphodiester bonds, although in some cases, nucleic acid analogs are included that may have at least one different linkage, e.g., phosphoramidate, phosphorothioate, phosphorodithioate, or O-methylphosphoroamidite linkages. The term nucleic acid may, in some contexts, be used interchangeably with gene, cDNA, mRNA, oligonucleotide, and polynucleotide.

A particular nucleic acid sequence also encompasses conservatively modified variants thereof (such as degenerate codon substitutions) and complementary sequences, as well as the sequence explicitly indicated. Specifically, degenerate codon substitutions may be achieved by generating sequences in which the third (“wobble”) position of one or more selected (or all) codons is substituted with mixed-base and/or deoxyinosine residues. Thus a nucleic acid sequence encoding a protein sequence disclosed herein also encompasses modified variants thereof as described herein.

The terms “polypeptide”, “peptide”, and “protein” are typically used interchangeably herein to refer to a polymer of amino acid residues. Amino acids may be referred to herein by either their commonly known three letter symbols or by the one-letter symbols recommended by the IUPAC-IUB Biochemical Nomenclature Commission.

As used herein, the terms “sample” and “biological sample” refer to any sample suitable for the methods provided by the present invention. In preferred embodiments, the sample contains nucleic acid and/or protein. In one embodiment, the biological sample of the present invention is a tissue sample, e.g., a biopsy specimen such as samples from needle biopsy, core needle biopsy or excisional biopsy (i.e., biopsy sample). In other embodiments, the biological sample of the present invention is a sample of bodily fluid, e.g., blood, serum, plasma, sputum, lung aspirate, or urine.

As used herein, the term “amplification” when used in reference to a gene or amplicon means a log 2(ratio) >1, in other words, the amplification event results in at least twice as many copies of the gene or the amplicon. As used herein, the term “gain” typically refers to a low level increase in copy number (i.e., less than a 2-fold increase).

Reference herein to “normal cells” or “corresponding normal cells” means cells that are from the same organ and of the same type as the cancer cell type. In one aspect, the corresponding normal cells comprise a sample of cells obtained from a healthy individual. Such corresponding normal cells can, but need not be, from an individual that is age-matched and/or of the same gender as the individual providing the cancer cells being examined. In another aspect, the corresponding normal cells comprise a sample of cells obtained from an otherwise healthy portion of tissue of a subject having cancer. In some embodiments of the present methods, the determination of a genomic gain is made by comparison of the genome from a cancer or tumor sample to a normal cell.

Cancers

It is contemplated that inhibitors of CDK4/6 or cyclin D are useful to treat cancers determined to be sensitive to treatment with a CDK4/6 or cyclin D inhibitor. It is contemplated that the methods herein identify cancers that are sensitive or resistant to CDK4/6 and/or cyclin D inhibitors. Exemplary cancers include but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, including triple negative breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor. In some embodiments, the cancer is selected from the group consisting of melanoma, colorectal cancer, pancreatic cancer, bladder cancer, breast cancer, triple negative breast cancer, ovarian cancer and lung cancer.

Breast Cancer

The human epidermal growth factor receptor (HER) family consists of four homologous receptors: epidermal growth factor receptor (EGFR/HER1), HER2, HER3, and HER4. All are transmembrane glycoproteins, but HER2 has no known ligand and HER3 has a nonfunctioning kinase. HER2 overexpression and/or amplification has been detected in 20% to 27% of invasive breast cancers and correlates with poorer clinical outcomes.

Triple-negative breast cancer (TNBC) refers to any breast cancer that does not express the genes for estrogen receptor (ER), progesterone receptor (PR) and Her2/neu. The lack of these markers makes it more difficult to treat since most chemotherapies target one of the three receptors, so triple-negative cancers often require combinatorial therapies. TNBC are sometimes classified into “basal-type” and other cancers. Basal type cancers are frequently defined by cytokeratin 5/6 and EGFR staining. About 75% of basal-type breast cancers are triple negative. Some TNBC overexpress epidermal growth factor receptor (EGFR) while some TNBC over expresses transmembrane glycoprotein NMB (GPNMB).

Melanoma

Malignant melanoma (MM) has long been considered a single histologic entity with heterogeneous clinical phenotypes. Evaluations of sun damage, mutations (v-raf murine sarcoma viral oncogene homolog B1 [BRAF], neuroblastoma RAS viral (v-ras) oncogene homolog [NRAS], and v-kit Hardy-Zuckerman 4 feline sarcoma viral oncogene homolog [CKIT]), and gene polymorphisms (melanocortin 1 receptor [MC1R]) indicate that melanoma is actually composed of distinct clinical and molecular entities driven by various oncogenic events (Tap et al, Neoplasia. 2010; 12(8): 637-649; Curtin et al., N Engl J Med. 2005; 353:2135-2147; Curtin et al., J Clin Oncol. 2006; 24:4340-4346; Landi et al., Science. 2006; 313:521-522). The identification of these molecular alterations permits the development of targeted therapies tailored to the specific pathway lesions of individual tumors. Mixed clinical outcome for therapies emphasizes the diverse nature of melanoma, questions its dependence on single “oncogenic” events, and demonstrates the complexity inherent to and of targeting molecular pathways.

The mitogen-activated protein kinase (MAPK) pathway is implicated in the pathogenesis and propagation of melanoma because of mutations in transmembrane receptor tyrosine kinases (RTKs) (CKIT 3%-5%), membrane-associated guanine nucleotide binding proteins (NRAS 15%-20%), and cytoplasmic serine/threonine kinases (BRAF 60%-75%). Collectively, the effect of these aberrant signaling mediators is the steady-state activation of extracellular signal-regulated kinase (ERK) observed in 90% of melanomas (Cohen et al., Clin Cancer Res. 2002; 8:3728-3733; Gray-Schopfer et al., Nature. 2007; 445:851-857). Regardless of the causative upstream event, ERK overexpression promotes the differentiation, malignant transformation, proliferation, and survival of MM. Specific to ERK activation in BRAF mutated melanoma, as opposed to RTK mediated ERK activation, is its resistance to negative feedback inhibition from the dual specific phosphatases (DUSP) and sprouty family (SPRY) of RAF binding proteins (Pratilas et al., Proc Natl Acad Sci USA. 2009; 106:4519-4524). Also, the presence of a BRAF (B-RAF) mutation (BRAF^{m}) seems to evoke downstream transcriptional activity of ERK through MYC, FOS-like antigen 1 (FOSL1), and the ETS family of transcription factors. Activation of the phosphoinositide-3-kinase (PI3K) pathway is documented in more than 70% of MM (Kantrow et all. J Cutan Pathol. 2007; 34:593-596). Synergistic activity of BRAF and v-akt murine thymoma viral oncogene homolog (AKT) has been implicated in the pathogenesis and malignant transformation of melanoma (Cheung et al., Cancer Res. 2008; 68:3429-3439; Dankort et al., Nat Genet. 2009; 41:544-552).

In various embodiments, the disclosure provides a method for treating a subject having melanoma that is sensitive to a CDK4 inhibitor, CDK6 inhibitor, or cyclin D inhibitor comprising identifying a subject having a CDK4 inhibitor-, CDK6 inhibitor-, or cyclin D inhibitor-sensitive melanoma and administering a CDK4 inhibitor, CDK6 inhibitor, or cyclin D inhibitor. In various embodiments, the CDK4 inhibitor-, CDK6 inhibitor-, or cyclin D inhibitor-sensitive melanoma comprises i) decrease in phospho-Rb1 protein; ii) decrease in FOXM1 protein; or iii) combinations of i) and ii). In various embodiments, the CDK4 inhibitor-, CDK6 inhibitor-, or cyclin D inhibitor-sensitive melanoma does not comprise i) activation of SHH; ii) overexpression of the SMO gene; iii) overexpression of the GL12 gene; or iv) combinations of i), ii) and iii). In various embodiments, a CDK4 inhibitor-, CDK6 inhibitor-, or cyclin D inhibitor-sensitive melanoma comprises i) activation of SHH; ii) overexpression of the SMO gene; iii) overexpression of the GL12 gene; or iv) combinations of i), ii) and iii).

Colon Cancer

Colon cancer typically originates from the epithelial cells lining the colon of the gastrointestinal tract. Certain mutations have been linked to colon cancer, including mutations in the Wnt signaling pathway that increase signaling activity. Other mutations that have been noted in colon cancers include p53 mutations and BAX mutations. Zhao et al., (World J Gastroenterol. 2003 October; 9(10):2202-6) disclosed that immunostaining of p16 was a predictor for better prognosis whereas strong cytoplasmic immunostaining of CDK4 was a predictor for poor prognosis. Certain studies have shown that administration of CDK4/6 inhibitor LY2835219 to patients has been beneficial in colon cancer.

In various embodiments, the disclosure contemplates a method for treating a subject having colon cancer that is sensitive to a CDK4 inhibitor, CDK6 inhibitor, or cyclin D inhibitor comprising identifying a subject having a CDK4 inhibitor-, CDK6 inhibitor-, or cyclin D inhibitor-sensitive colon cancer and administering a CDK4 inhibitor, CDK6 inhibitor, or cyclin D inhibitor. In various embodiments, the CDK4 inhibitor-, CDK6 inhibitor-, or cyclin D inhibitor-sensitive colon cancer comprises i); high p16 protein; ii) copy number amplification of RB1; or iii) combinations of i) and ii).

In various embodiments, the CDK4 inhibitor-, CDK6 inhibitor-, or cyclin D inhibitor-sensitive colon cancer does not comprise i) loss-of-function mutations in FBXW7, ii) point mutations in RET; iii) CCNE1 copy number amplifications, iv) or combinations of any of i), ii) or iii). In various embodiments, a CDK4 inhibitor-, CDK6 inhibitor-, or cyclin D inhibitor-resistant colon cancer comprises, i) loss-of-function mutations in FBXW7, ii) point mutations in RET; iii) CCNE1 copy number amplifications, iv) or combinations of any of i), ii) or iii).

Gastric Cancer

In various embodiments, the disclosure contemplates a method for treating a subject having gastric cancer that is sensitive to a CDK4 inhibitor, CDK6 inhibitor, or cyclin D inhibitor comprising identifying a subject having a CDK4 inhibitor-, CDK6 inhibitor-, or cyclin D inhibitor-sensitive gastric cancer and administering a CDK4 inhibitor, CDK6 inhibitor, or cyclin D inhibitor. In various embodiments, the CDK4 inhibitor, CDK6 inhibitor, or cyclin D inhibitor-sensitive gastric cancer comprises i) CCND1 amplification; ii) ERBB2 amplification; or iii) combinations of i) and ii). In various embodiments, the CDK4 inhibitor-, CDK6 inhibitor-, or cyclin D inhibitor-sensitive gastric cancer does not comprise i) loss-of-function mutations in FBXW7, ii) loss-of-function mutations in RB1; iii) CCNE1 copy number amplification; or iv) combinations of any of i), ii) or iii). In various embodiments, a CDK4 inhibitor-, CDK6 inhibitor-, or cyclin D inhibitor-resistant gastric cancer comprises, i) loss-of-function mutations in FBXW7, ii) loss-of-function mutations in RB1; iii) CCNE1 copy number amplification; or iv) combinations of any of i), ii) or iii).

Assays

Malignant tissue specimens of cancers from individual patients or circulating tumor cells could be tested for the presence of alterations in the relevant predictive genes biomarkers of sensitivity/resistance by any of the methods listed above. If the types of alterations listed in this disclosure are found to be present, these patients could be considered as appropriate or inappropriate candidates to receive CDK4 and/or CDK6 or Cyclin D-inhibitor based therapies as part of the treatment regimen for their malignancy.

The in vitro response biomarkers described herein can be translated for use in the clinical setting via genotyping the patient tumor samples for each particular alteration. The assays used to perform this genotyping would differ by alteration type. Copy number alterations can be assayed by several techniques including (but not limited to) SNP arrays, comparative genomic hybridization (CGH), southern blot analysis, florescent in-situ hybridization (FISH) or chromogenic in situ hybridization (CISH) in order to detect increases in DNA copy number at these loci. To detect the alterations at the RNA level, this would include (but not be limited to) techniques such as transcript expression arrays, RNA in situ hybridization, northern blot analysis, transcript enumeration via direct exon/transcript sequencing (e.g. Illumina sequencing platforms) in order to detect increases in mRNA expression of the gene transcripts. To detect the alterations at the protein level, this would include (but not be limited to) techniques such as protein arrays (e.g. ELISAs, Multiplex (MSD), reverse phase protein analysis-RPPA or western blot analysis of cell or tissue lysates/extracts, etc.) immunohistochemical staining analyses of tissue sections for the presence of the target proteins, or any antibody-based methodology directed at detecting increases in protein expression of the target proteins. Point mutations can be assayed directly by Sanger sequencing, or next-generation array based methods such as hybrid capture. Point mutations may also be assayed indirectly at the mRNA or protein level if the alteration leads to instability of the mRNA transcript or degradation of the resulting protein.

Additional assays are described in the Examples section.

The biomarkers identified in the assays are referred to be their common name known in the literature and can be identified using literature or other common scientific resources.

Formulations

The disclosure provides CDK4/6 and cyclin D inhibitors useful in the treatment of cancer (e.g., to inhibit or suppress growth of or metastasis of a tumor). To administer inhibitors to patients or test animals, it is preferable to formulate the products in a composition comprising one or more pharmaceutically acceptable carriers. Pharmaceutically or pharmacologically acceptable carriers or vehicles refer to molecular entities and compositions that do not produce allergic, or other adverse reactions when administered using routes well-known in the art, as described below, or are approved by the U.S. Food and Drug Administration or a counterpart foreign regulatory authority as an acceptable additive to orally or parenterally administered pharmaceuticals. Pharmaceutically acceptable carriers include any and all clinically useful solvents, dispersion media, coatings, antibacterial and antifungal agents, isotonic and absorption delaying agents and the like.

Pharmaceutical carriers include pharmaceutically acceptable salts, particularly where a basic or acidic group is present in a compound. For example, when an acidic substituent, such as —COOH, is present, the ammonium, sodium, potassium, calcium and the like salts, are contemplated for administration. Additionally, where an acid group is present, pharmaceutically acceptable esters of the compound (e.g., methyl, tert-butyl, pivaloyloxymethyl, succinyl, and the like) are contemplated as preferred forms of the compounds, such esters being known in the art for modifying solubility and/or hydrolysis characteristics for use as sustained release or prodrug formulations.

When a basic group (such as amino or a basic heteroaryl radical, such as pyridyl) is present, then an acidic salt, such as hydrochloride, hydrobromide, acetate, maleate, pamoate, phosphate, methanesulfonate, p-toluenesulfonate, and the like, is contemplated as a form for administration.

In addition, compounds may form solvates with water or common organic solvents. Such solvates are contemplated as well.

The inhibitors herein may be administered orally, parenterally, transocularly, intranasally, transdermally, transmucosally, by inhalation spray, vaginally, rectally, or by intracranial injection. The term parenteral as used herein includes subcutaneous injections, intravenous, intramuscular, intracisternal injection, or infusion techniques. Administration by intravenous, intradermal, intramusclar, intramammary, intraperitoneal, intrathecal, retrobulbar, intrapulmonary injection and or surgical implantation at a particular site is contemplated as well. Generally, compositions for administration by any of the above methods are essentially free of pyrogens, as well as other impurities that could be harmful to the recipient. Further, compositions for administration parenterally are sterile.

Inhibitors contemplated herein include, but are not limited to CDK4/6 and cyclin D inhibitors palbociclib, LY2835219, and LEE011.

Dosing and Administration

The CDK4/6 or cyclin D inhibitor is administered in a therapeutically effective amount; typically, the composition is in unit dosage form. The amount of inhibitor administered is, of course, dependent on the age, weight, and general condition of the patient, the severity of the condition being treated, and the judgment of the prescribing-physician. Suitable therapeutic amounts will be known to those skilled in the art and/or are described in the pertinent reference texts and literature. In one aspect, the dose is administered either one time per day or multiple times per day. The CDK4/6 or cyclin D inhibitor may be administered one, two, three or four times per day. In some embodiments, an effective dosage of inhibitor may be within the range of 0.01 mg to 1000 mg per kg (mg/kg) of body weight per day. In some embodiments, the inhibitor is administered at a daily dose ranging from about 10 mg/kg to about 250 mg/kg, or from about 100 mg/kg to about 250 mg/kg, or from about 60 mg/kg to about 100 mg/kg or from about 50 mg/kg to about 90 mg/kg, or from about 30 mg/kg to about 80 mg/kg, or from about 20 mg/kg to about 60 mg/kg, or from about 10 mg/kg to about 50 mg/kg. Further, the effective amount or dose may be 0.5 mg/kg, 1 mg/kg, 5 mg/kg, 10 mg/kg, 15 mg/kg, 20 mg/kg/25 mg/kg, 30 mg/kg, 35 mg/kg, 40 mg/kg, 45 mg/kg, 50 mg/kg, 55 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, 250 mg/kg, 300 mg/kg, 350 mg/kg, 400 mg/kg, 450 mg/kg, 500 mg/kg, and may increase by 25 mg/kg increments up to 1000 mg/kg, or may range between any two of the foregoing values.

Administration may continue for at least 1 month, 2 months, 3 months, 6 months, 9 months, 1 year, 2 years, or more.

Combination Therapy

Therapeutic compositions described herein can be administered in therapeutically effective dosages alone or in combination with adjunct cancer therapy such as surgery, chemotherapy, radiotherapy, thermotherapy, and laser therapy, and may provide a beneficial effect, e.g. reducing tumor size, slowing rate of tumor growth, inhibiting metastasis, or otherwise improving overall clinical condition, without necessarily eradicating the cancer. Cytostatic and cytotoxic agents that target the cancer cells are specifically contemplated for combination therapy. Likewise, agents that target angiogenesis or lymphangiogenesis are specifically contemplated for combination therapy.

Traditional approaches to the design of combination therapeutic regimens have involved adding a new drug to an already established regimen, assuming that there is no increased toxicity caused by integrating the new drug. It is contemplated that a CDK4/6 or cyclin D inhibitor can be used in combination with an already established standard of care for a particular cancer, or in another combination regimen. The present methods may also indicate that a CDK4/6 or cyclin D inhibitor is contraindicated in combination therapy with a standard of care treatment if the cancer is determined to be resistant to a CDK4/6 or cyclin D inhibitor. See e.g., Pegram et al., J Natl Cancer Inst (2004) 96(10):759-769).

As used herein, a “chemotherapeutic agent” is a chemical compound useful in the treatment of cancer. Examples of chemotherapeutic agents include: alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and tiimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; vinca alkaloids; epipodophyllotoxins; antibiotics such as the enediyne antibiotics (e.g., calicheamicin, especially calicheamicin gammall and calicheamicin omegall; L-asparaginase; anthracenedione substituted urea; methyl hydrazine derivatives; dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitiaerine; pentostatin; phenamet; pirarubicin; losoxantione; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2 2″-trichlorotiiethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® (docetaxel) (Rhone-Poulenc Rorer, Antony, France); chloranbucil; GEMZAR® (gemcitabine); 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DFMO); retinoids such as retinoic acid; capecitabine; leucovorin (LV); irenotecan; adrenocortical suppressant; adrenocorticosteroids; progestins; estrogens; androgens; gonadotropin-releasing hormone analogs; and pharmaceutically acceptable salts, acids or derivatives of any of the above. Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON-toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASL® exemestane, formestanie, fadrozole, RIVISOR® vorozole, FEMARA® letrozole, and ARTMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF-A expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rJL-2; LURTOTECAN® topoisomerase 1 inhibitor; ABARELLX® rmRH; antibodies such as trastuzumab and pharmaceutically acceptable salts, acids or derivatives of any of the above.

The treatment methods described herein optionally include monitoring the effect of the therapeutic composition on the tumor. For example, the size of the tumor can be determined, as can the presence of metastases. Also contemplated is measurement of the degree of metastasis, e.g., by measuring the number of metastatic modules or by measurement of ascites associated with metastasis.

The CDK4/6 or cyclin D inhibitor and other drugs/therapies can be administered in combination either simultaneously in a single composition or in separate compositions. Alternatively, the administration is sequential. Simultaneous administration is achieved by administering a single composition or pharmacological protein formulation that includes both the inhibitor and other therapeutic agent(s). Alternatively, the other therapeutic agent(s) are taken separately at about the same time as a pharmacological formulation (e.g., tablet, injection or drink) of the inhibitor.

Kits

The disclosure also provides kits for carrying out the methods of the disclosure. In various embodiments, the kit contains, e.g., bottles, vials, ampoules, tubes, cartridges and/or syringes that comprise a liquid (e.g., sterile injectable) formulation or a solid (e.g., lyophilized) formulation. The kits can also contain pharmaceutically acceptable vehicles or carriers (e.g., solvents, solutions and/or buffers) for reconstituting a solid (e.g., lyophilized) formulation into a solution or suspension for administration (e.g., by injection), including without limitation reconstituting a lyophilized formulation in a syringe for injection or for diluting concentrate to a lower concentration. Furthermore, extemporaneous injection solutions and suspensions can be prepared from, e.g., sterile powder, granules, or tablets comprising a composition comprising an inhibitor as described herein. The kits can also include dispensing devices, such as aerosol or injection dispensing devices, pen injectors, autoinjectors, needleless injectors, syringes, and/or needles. In various embodiments, the kit also provides an oral dosage form, e.g., a tablet or capsule or other oral formulation described herein, of the inhibitor for use in the method. The kit also provides instructions for use.

While the disclosure has been described in conjunction with specific embodiments thereof, the foregoing description as well as the examples which follow are intended to illustrate and not limit the scope of the disclosure. Other aspects, advantages and modifications within the scope of the disclosure will be apparent to those skilled in the art.

Methods:

A cell line panel consisting of more than 600 human cancer cell lines derived from actual individual patient malignancies representing a broad spectrum of common human cancers that include 15 separate histologic subtypes, e.g. breast, ovary, lung, colorectal, gastric, melanoma, pancreas, etc. has been collected and comprehensively characterized. Specifically, this panel has been characterized with regard to the individual cell line's ability to grow in vitro both on plastic and in soft agar, as well as to grow in vivo subcutaneously and ortho-topically. In addition, each cell line in the panel has been characterized molecularly for gene expression by transcript microarray as well as gene copy number variation (CNV), and for many cell lines, by proteomic and biochemical pathway analyses. Finally, the majority of these cells have also been characterized for their response to standard chemotherapeutic/cytotoxic agents and where appropriate, hormonal or biologic agents. Using this characterized panel, preclinical, growth inhibition studies may be performed with various new potential therapeutics or novel combinations of therapeutics to determine which lines and/or histologies do or do not respond to the therapeutic intervention being assessed.

Using this research platform the present inventors have completed studies with palbociclib (i.e., PD-0332991), a novel cyclin dependent kinase 4/6 inhibitor. While not wishing to be bound by any particular theory, it is believed that blocking CDK 4/6 in cancer cells will inhibit the cell cycle at the G1/S transition by preventing the phosphorylation of the Rb gene product by the Cyclin D: CDK 4/6 enzyme complex.

The present inventors investigated potential molecular determinants of response to palbociclib in additional cancer types. As provided herein, the in vitro sensitivity to palbociclib was assayed across a panel of 470 cancer cell lines derived from 13 distinct cancer histologies. This analysis demonstrated a highly differential response to treatment both within and between cancer histologies. The IC50s (the concentration of palbociclib required to inhibit 50% of population doublings) ranged from the low nanomolar range to above the highest dose tested (1 μM). The generation of this highly differential response dataset allowed for a unique opportunity to explore the possible genetic mechanisms underlying differential sensitivity to treatment with palbociclib in vitro (which may help identify new and/or additional novel therapeutic targets) while at the same time allowing the identification of potential predictive biomakers of sensitivity and resistance.

Using this approach, two large genomic datasets were interrogated for genotype-response associations. The first dataset consisted of whole exome point mutation data downloaded from the Cancer Cell Line Encyclopedia's hybrid capture sequencing database. This database was restricted by various criteria to enrich for functional, somatic point mutations that are known to be causally involved in carcinogenesis. From this restricted dataset two proto-oncogenes (RET, SMO) were identified where presumptive “activating” mutations that were associated with resistance to treatment with palbociclib within the interrogated cell line panel.

As provided herein, eight recessive cancer genes where likely “loss-of-function” point mutations were significantly associated with palbociclib response were also identified. Mutations in one of these genes (CDH1) was associated with palbociclib sensitivity, while mutations in the seven other genes (RB1, FBXW7, FANCA, NBN, PMS2, STK11, MUTYH) were associated with resistance to the drug.

The second large genomic dataset that was interrogated for genotype-response associations was a copy number alteration dataset derived from comparative genomic hybridization arrays. This dataset was organized by gene and also restricted by various criteria to enrich for amplifications or deletions of genes likely to be causally involved in oncogenesis and/or tumor progression. From this dataset three chromosomal regions were identified (17q12-21, 11q13, 1q) where amplification was associated with sensitivity to palbociclib. Two amplified regions (19q12, 8q13) were found to be associated with resistance to the drug. Homozygous deletions of the 13q14 chromosomal region were found to be strongly associated with resistance to palbociclib.

Following the identification of these candidate palbociclib response biomarkers from the above screens, several analyses were performed to further validate causation for each of these genotype-response associations. These analyses included: a comprehensive literature search to investigate potential causal mechanisms for each identified biomarker; a Pearson correlation analysis to identify inter-biomarker associations followed by multiple regression to isolate the independent effect of each variable; a control of confounding variables by cell line growth rate and histology; and an analysis of misclassification in the genomics datasets. Using these extensive post-hoc analyses, the original set of fifteen biomarkers was reduced down to the eight most likely to play a causal role in determining sensitivity or resistance to treatment with palbociclib. The set of sensitivity biomarkers includes: loss-of-function point mutations in CDH1, chromosomal amplification of 17q12-21 (ERBB2), chromosomal amplification of 11q13 (CCND1) and chromosomal amplification of 1q (MDM4). The set of resistance biomarkers includes: an activating point mutation of the smoothened (SMO) gene, an activating point mutation of the RET proto-oncogene, a chromosomal amplification of 19q12 (CCNE1), a loss-of-function point mutation of FBXW7, a loss-of-function point mutation of retinoblastoma (RB1), a chromosomal deletion of RB1, a chromosomal amplification of sonic hedgehog (SHH), high baseline GLI2 mRNA expression, and high baseline SMO mRNA expression.

The above sets of biomarkers were analyzed by strata representing each of the twelve cancer types contained in the cell line panel. The four sensitivity biomarkers were sufficiently frequent and associated with sensitivity in 6 of 12 cancer types in our panel. These included breast, colon, head/neck, lung, ovarian and upper gastrointestinal malignancies. The resistance biomarkers were sufficiently frequent and associated with resistance in 6 of the 12 cancer types in our panel, which included breast, colon, kidney, lung, ovarian and upper gastrointestinal malignancies.

The in vitro response biomarkers described above can be translated for use in the clinical setting via genotyping the patient tumor samples for each particular alteration. The assays used to perform this genotyping would differ by alteration type. Copy number alterations can be assayed by several techniques including (but are not limited to) SNP arrays, comparative genomic hybridization (CGH), southern blot analysis or florescent in-situ hybridization (FISH) in order to detect increases in DNA copy number at these loci. To detect the alterations at the RNA level, this would include (but not be limited to) techniques such as transcript expression arrays, RNA in situ hybridization, real-time reverse transcription PCR (RT-PCR), northern blot analysis, transcript enumeration via direct exon/transcript sequencing (e.g. Lumina sequencing platforms) in order to detect increases in mRNA expression of the gene transcripts. To detect the alterations at the protein level, this would include (but not be limited to) techniques such as protein arrays (e.g. ELISAs, Multiplex (MSD), reverse phase protein analysis-RPPA or western blot analysis of cell or tissue lysates/extracts, etc.) immunohistochemical staining analyses of tissue sections for the present of the target proteins, or any antibody-based methodology directed at detecting increases in protein expression of the target proteins. Point mutations can be assayed directly by Sanger sequencing, or next-generation array based methods such as hybrid capture. Point mutations may also be assayed indirectly at the mRNA or protein level if the alteration leads to instability of the mRNA transcript or degradation of the resulting protein.

Malignant tissue specimens of cancers from individual patients or circulating tumor cells could be tested for the presence of alterations in the relevant predictive genes biomarkers of sensitivity/resistance by any of the methods listed above. If the types of alterations listed in this disclosure are found to be present, these patients could be considered as appropriate or inappropriate candidates to receive CDK4/6-inhibitor based therapies as part of the treatment regimen for their malignancy.

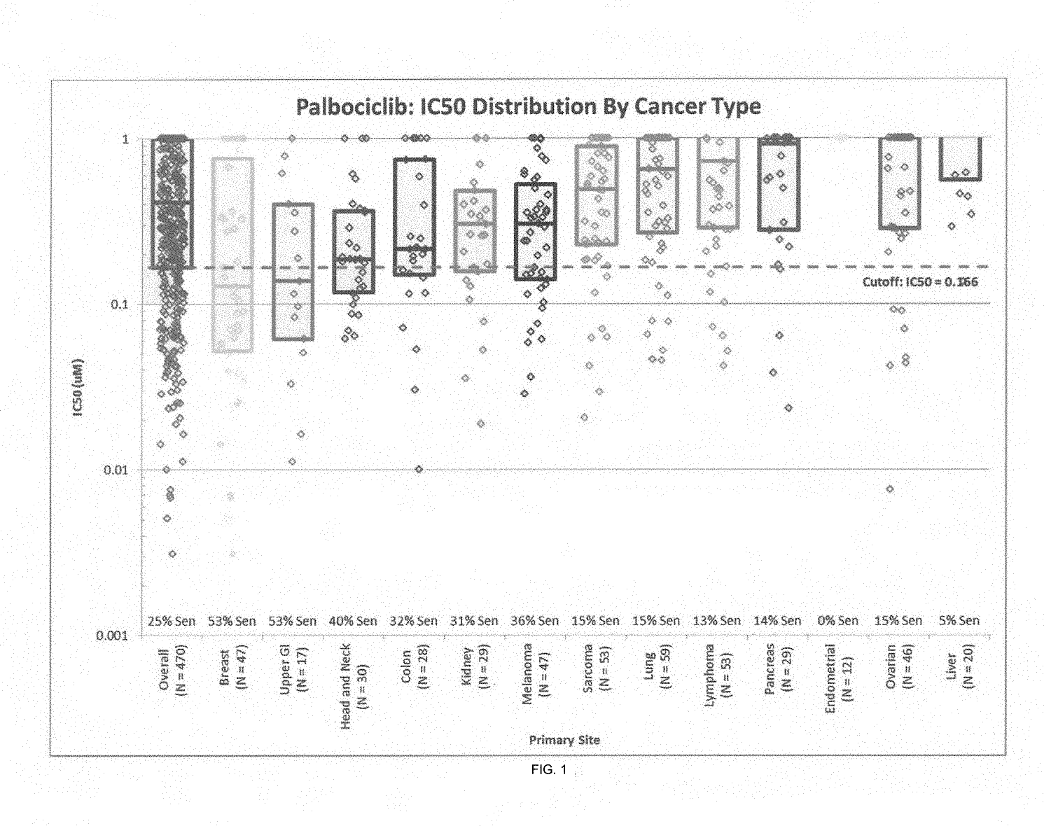

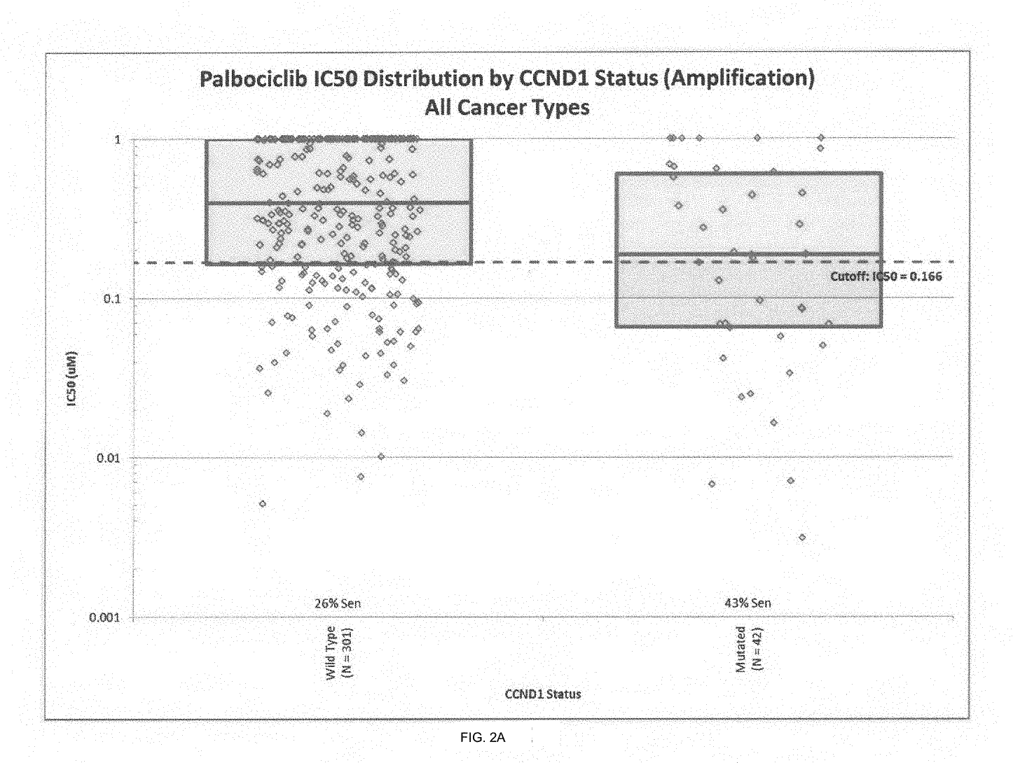

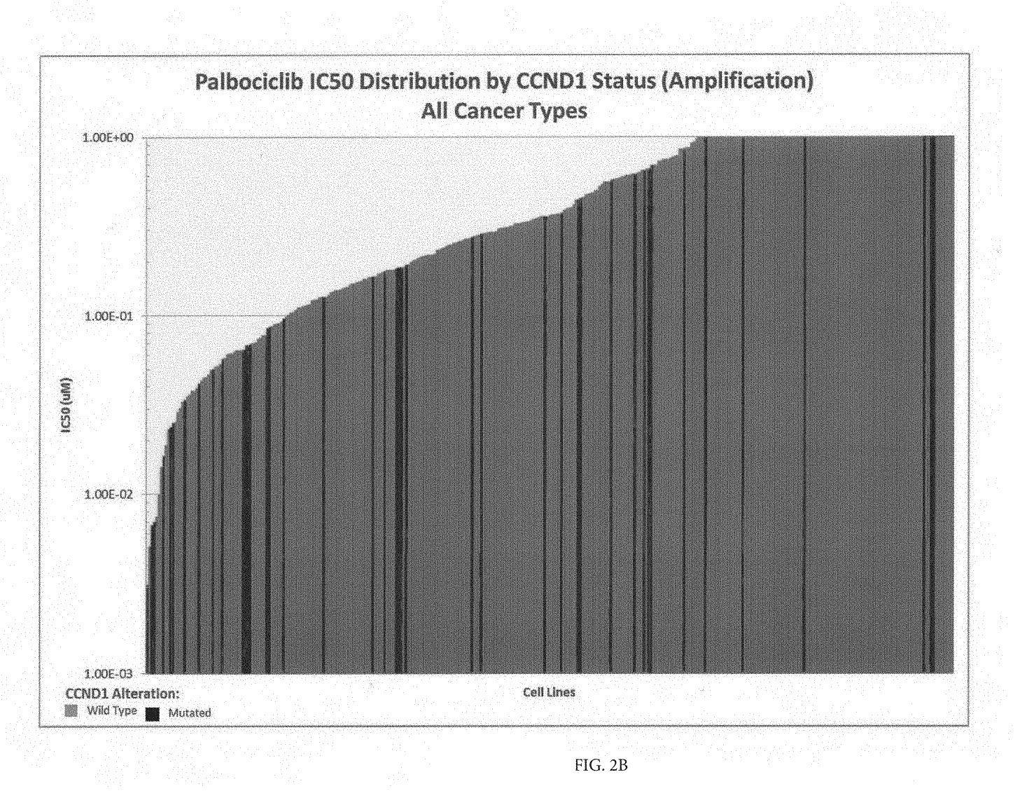

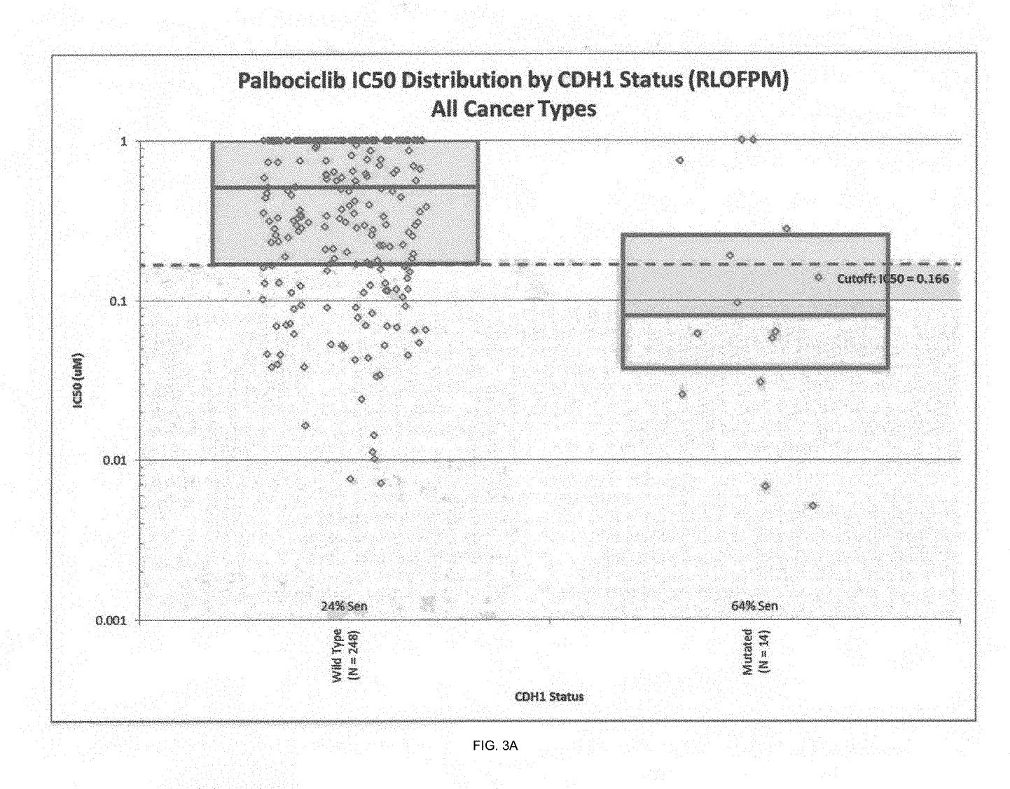

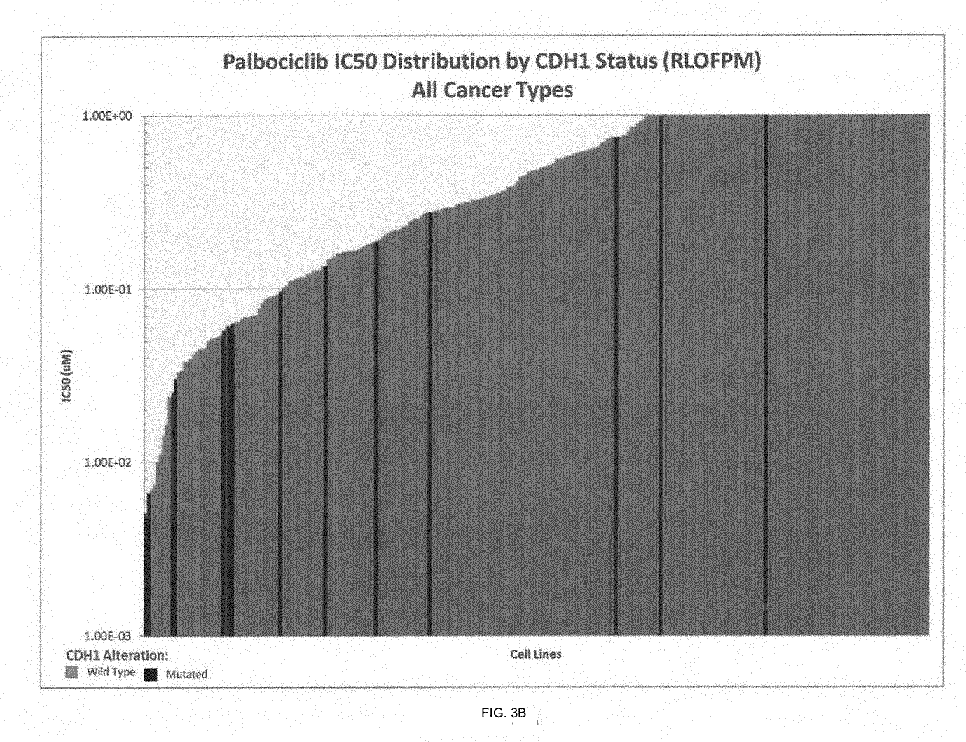

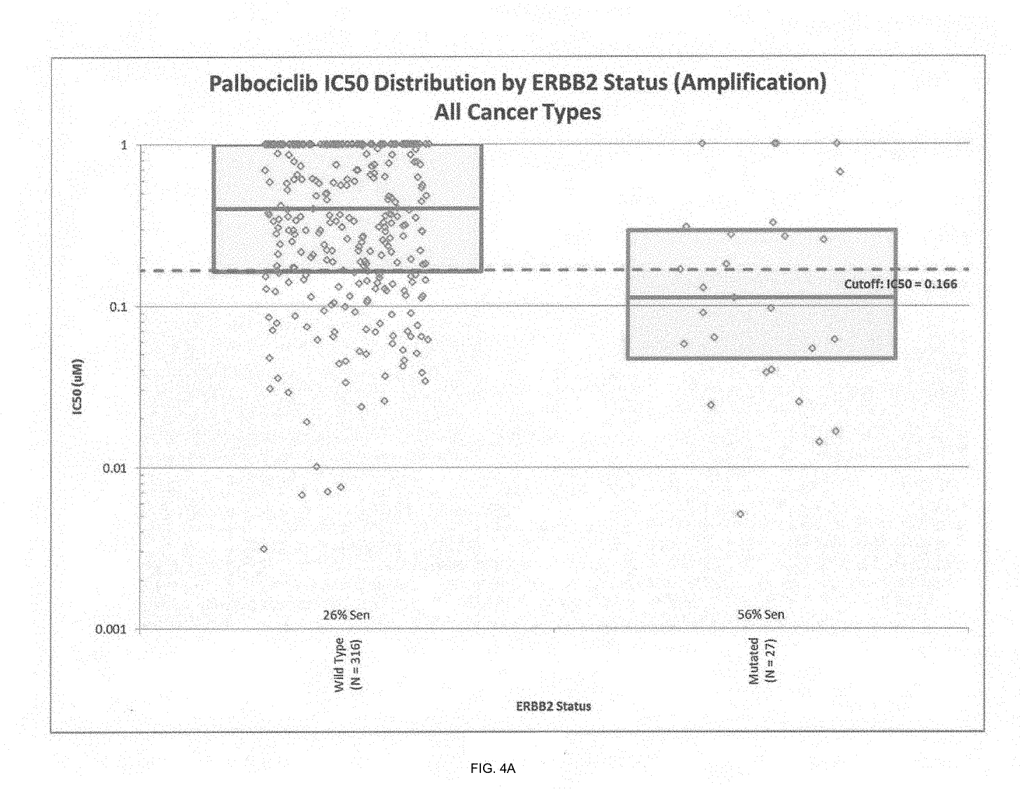

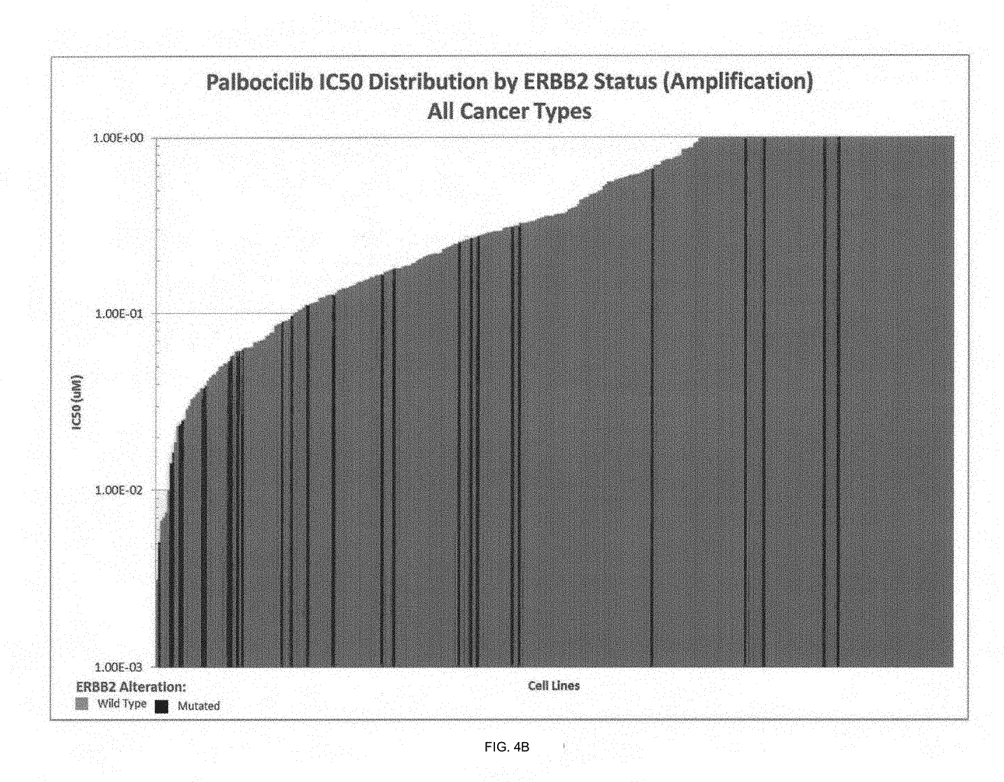

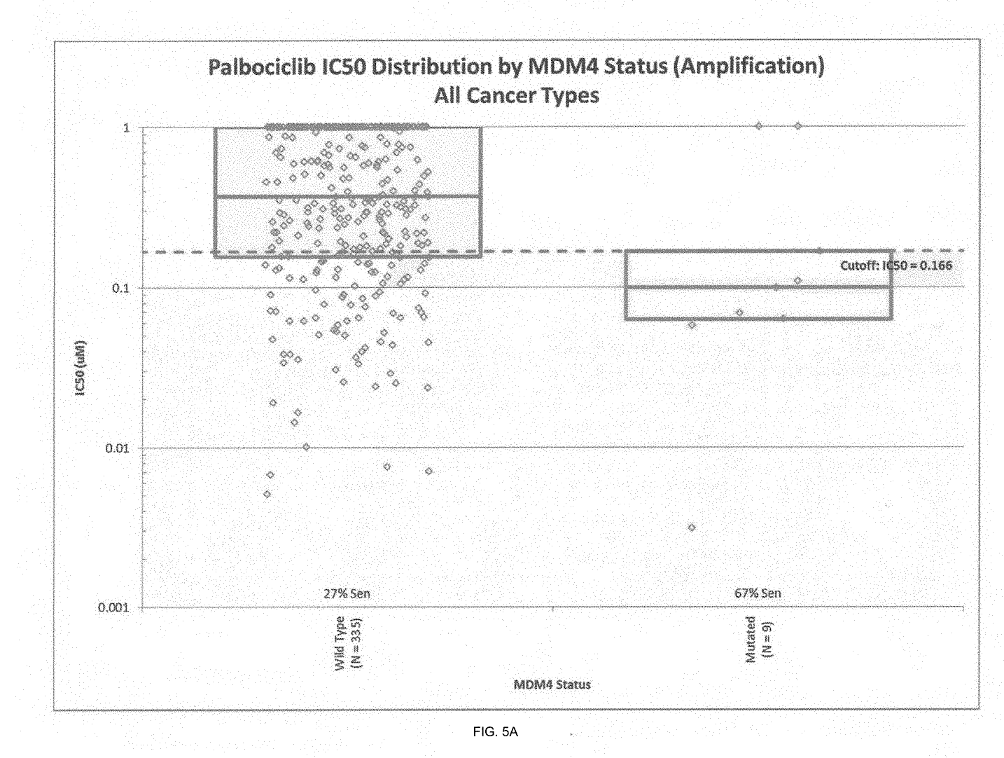

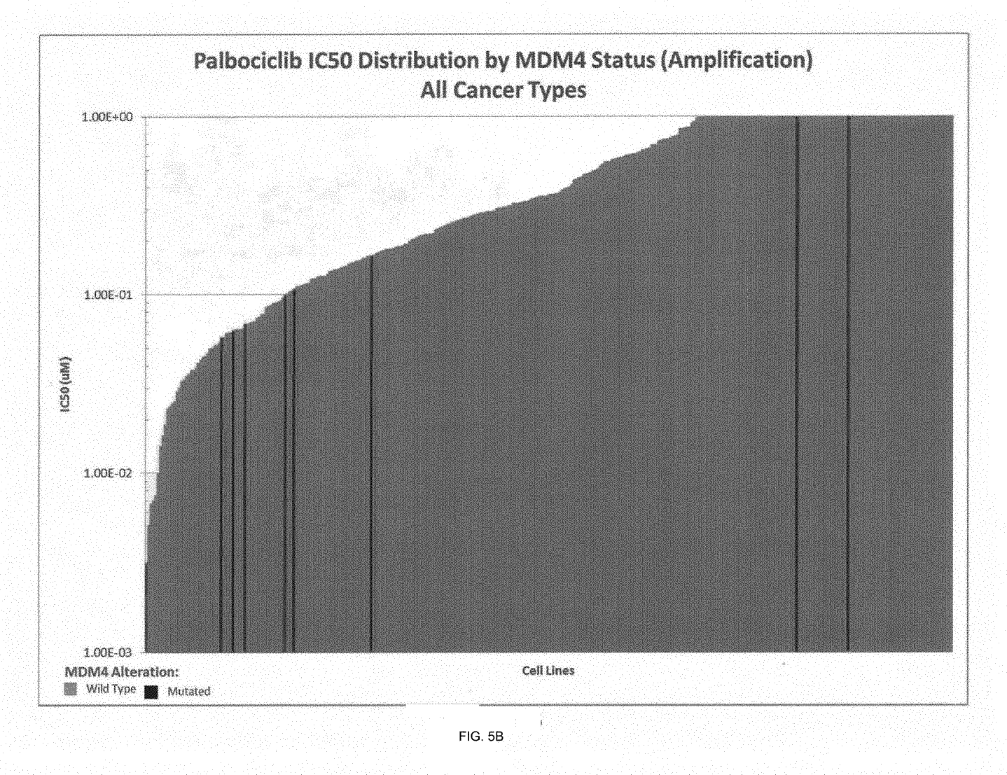

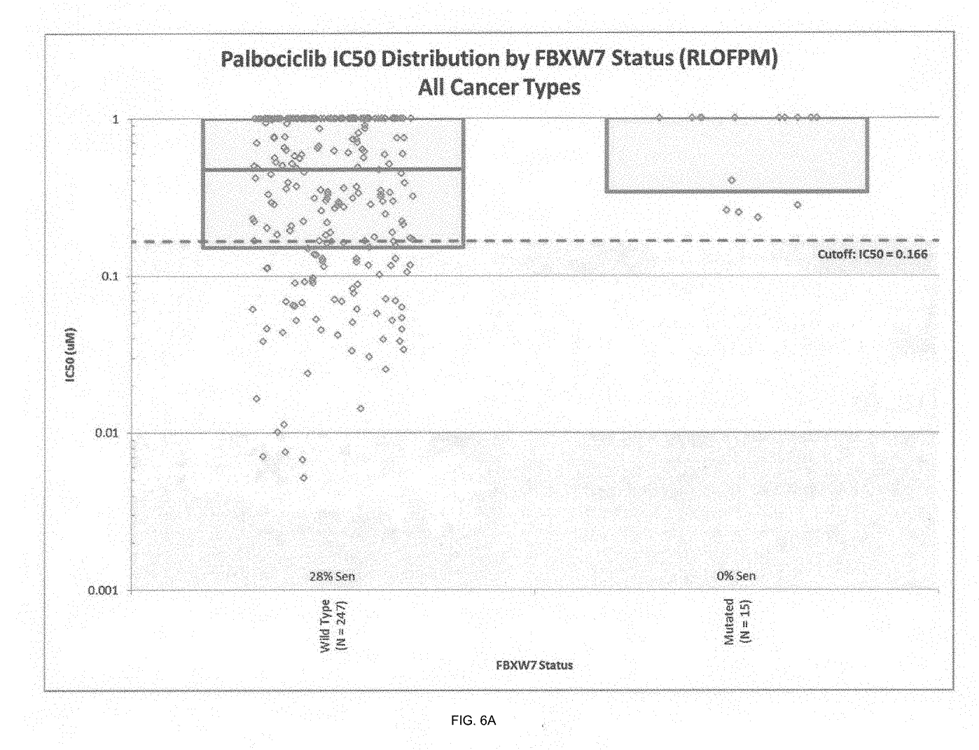

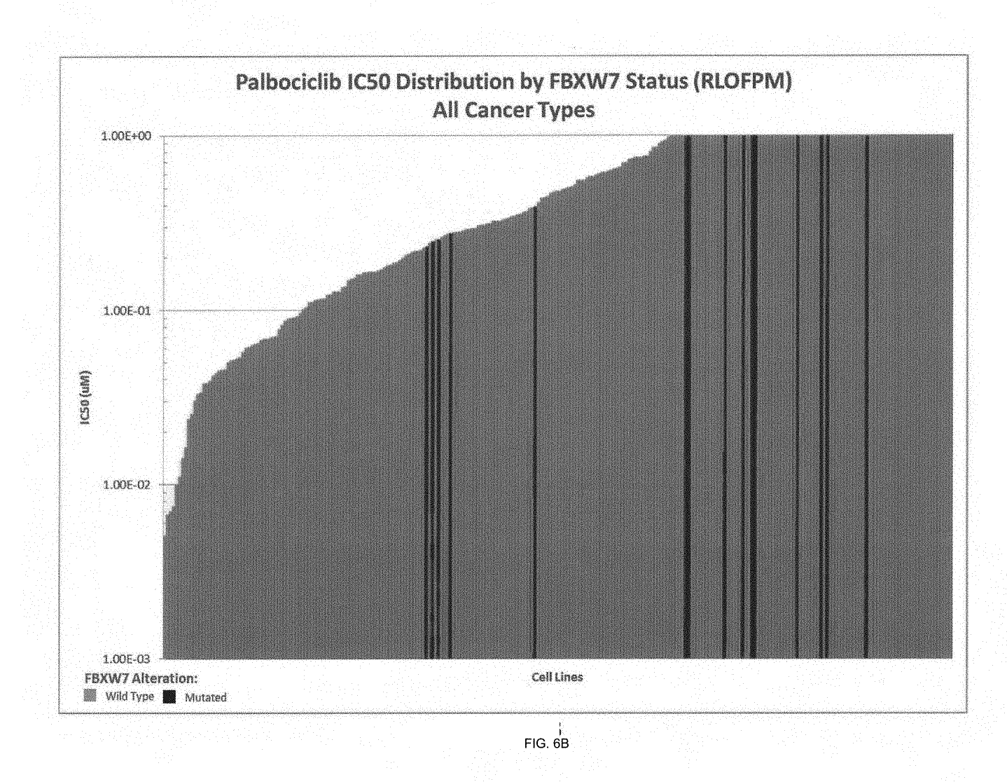

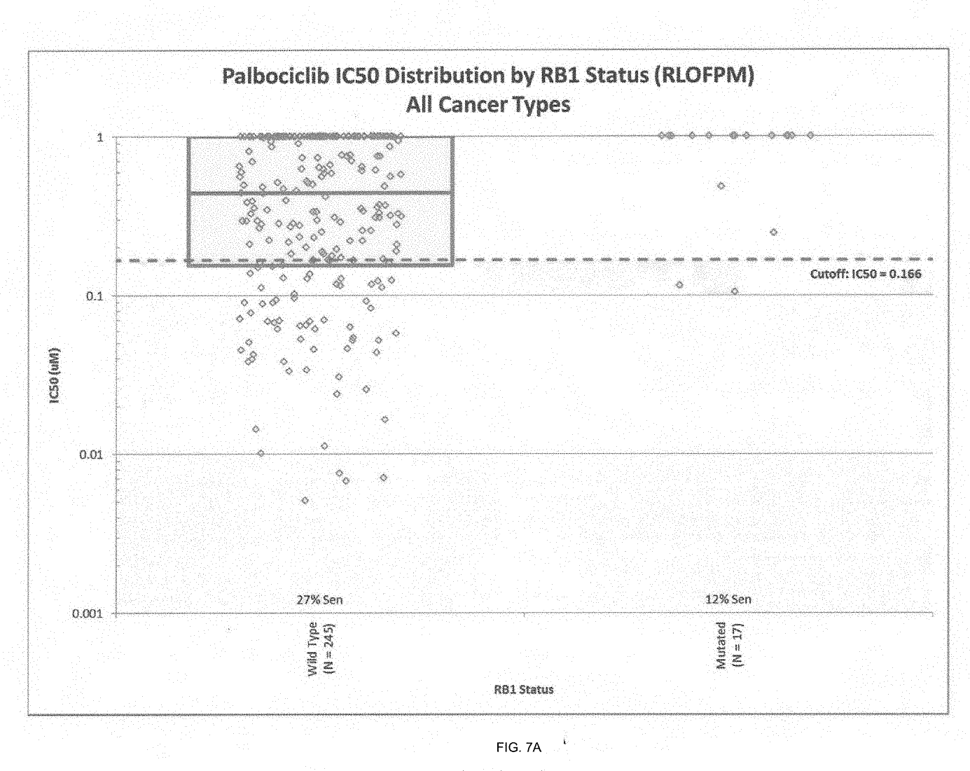

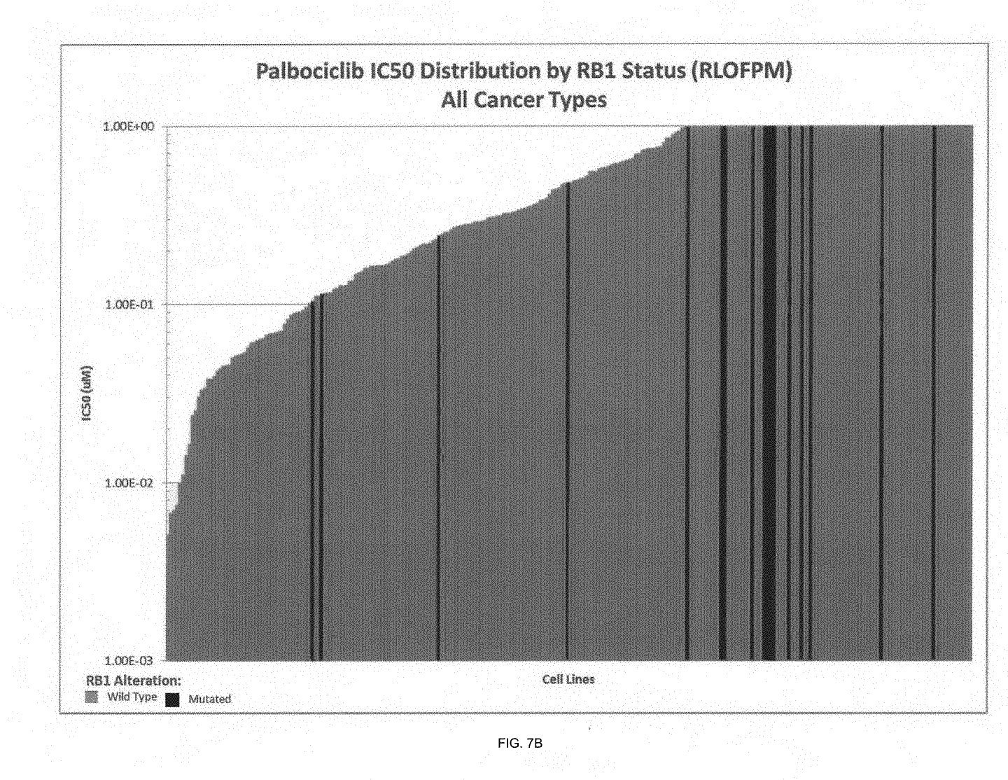

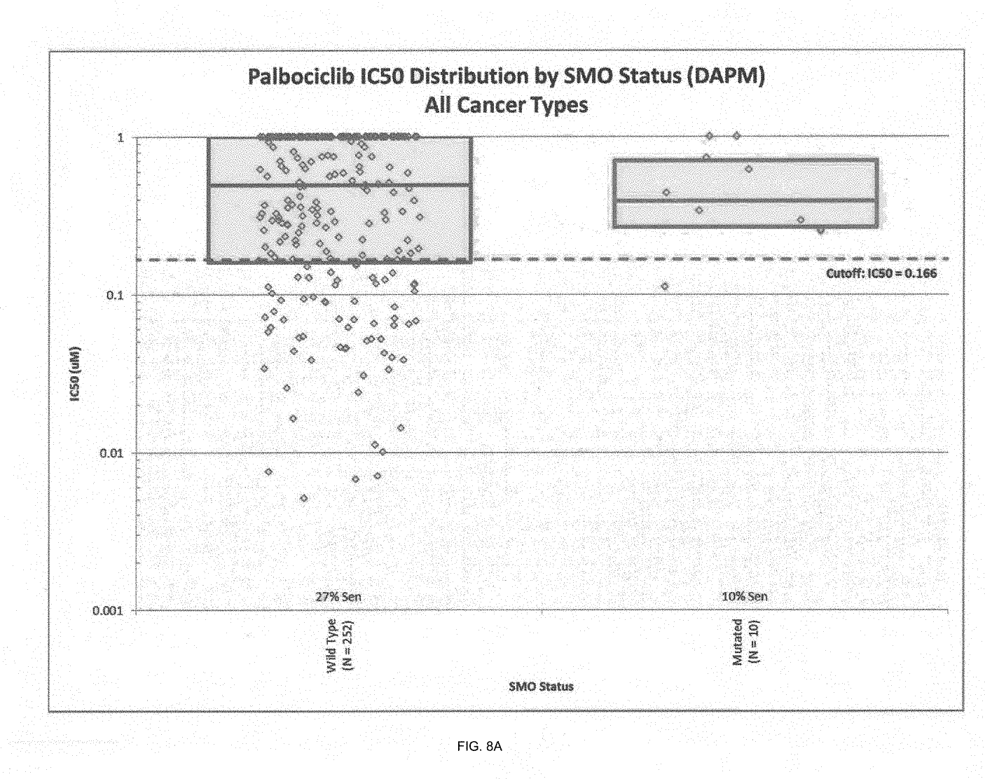

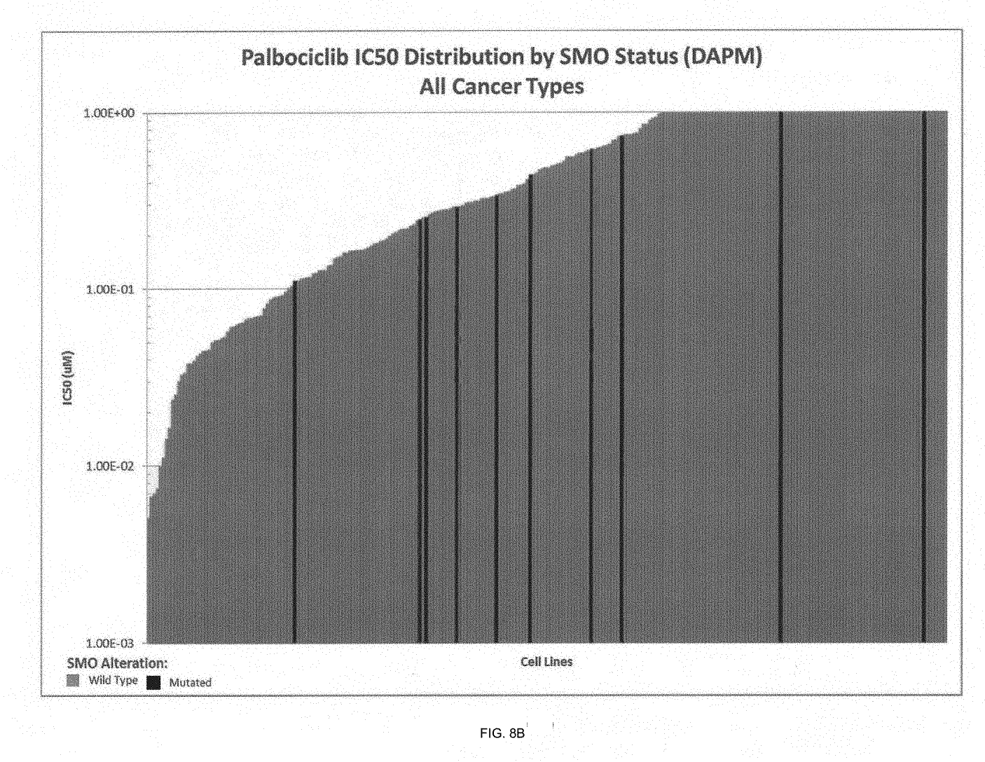

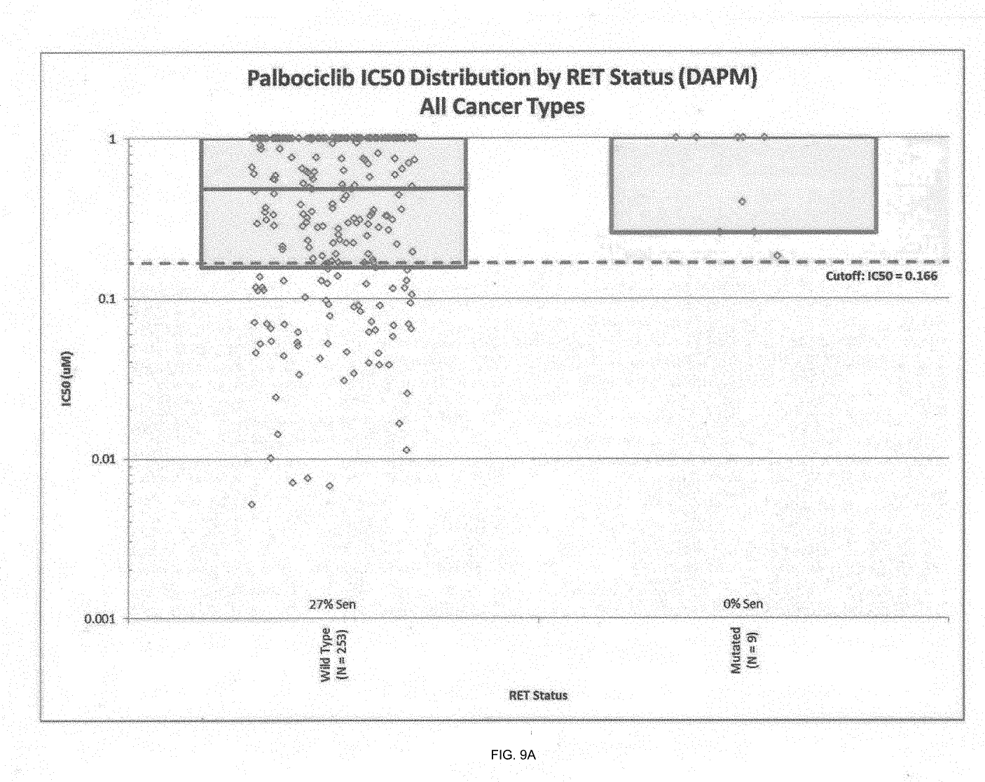

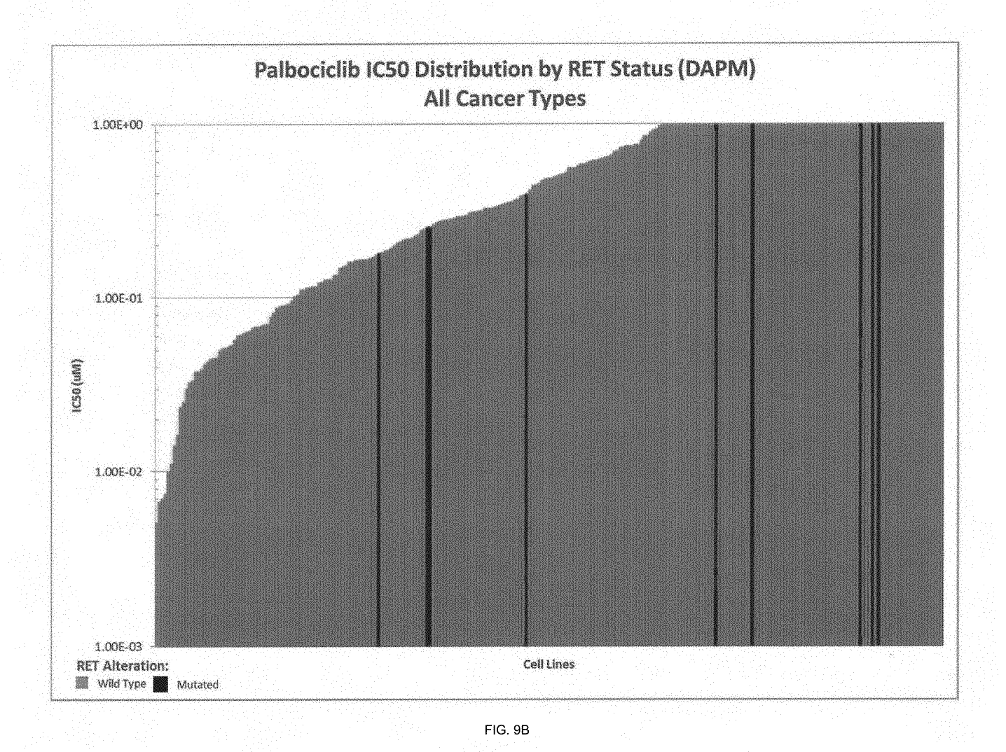

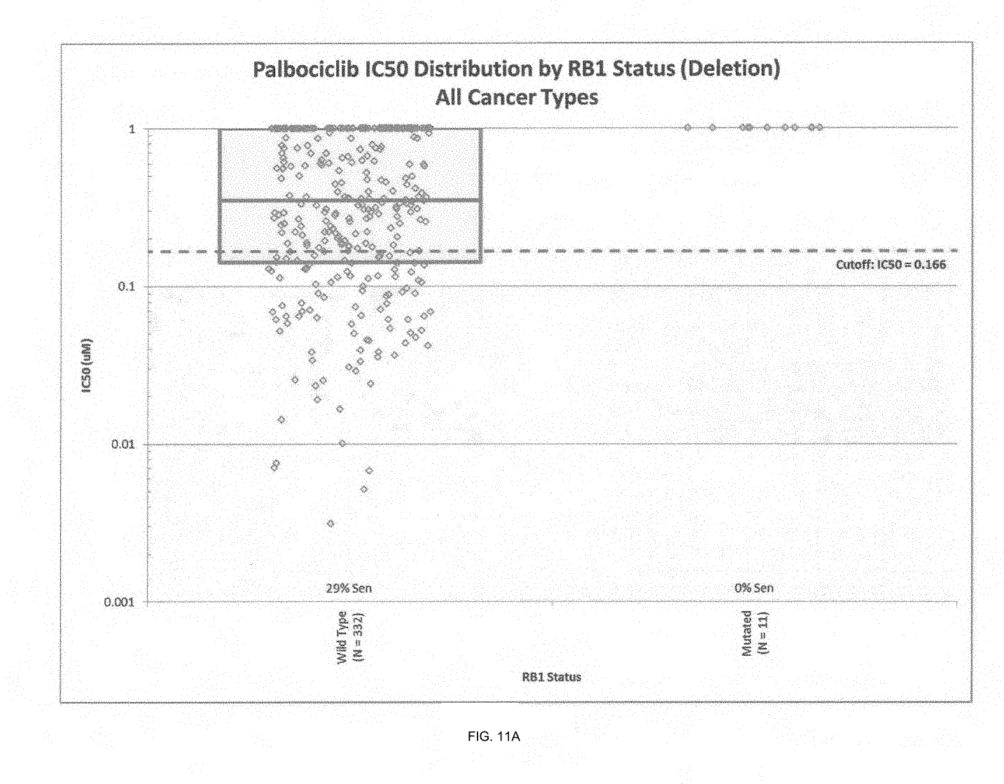

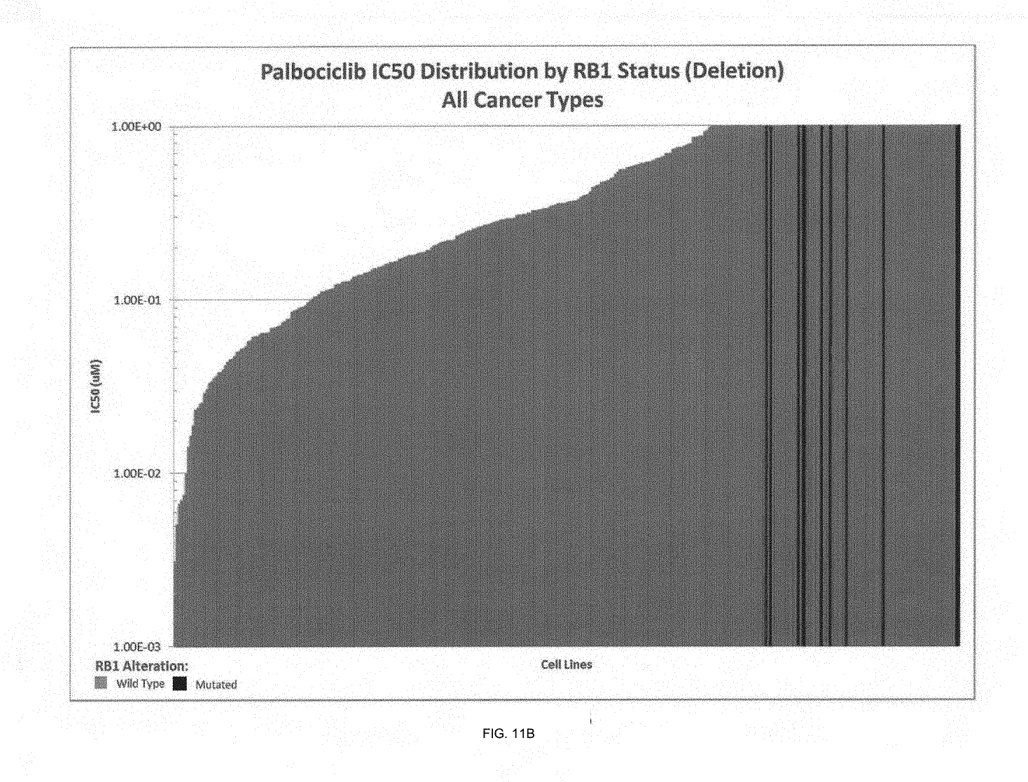

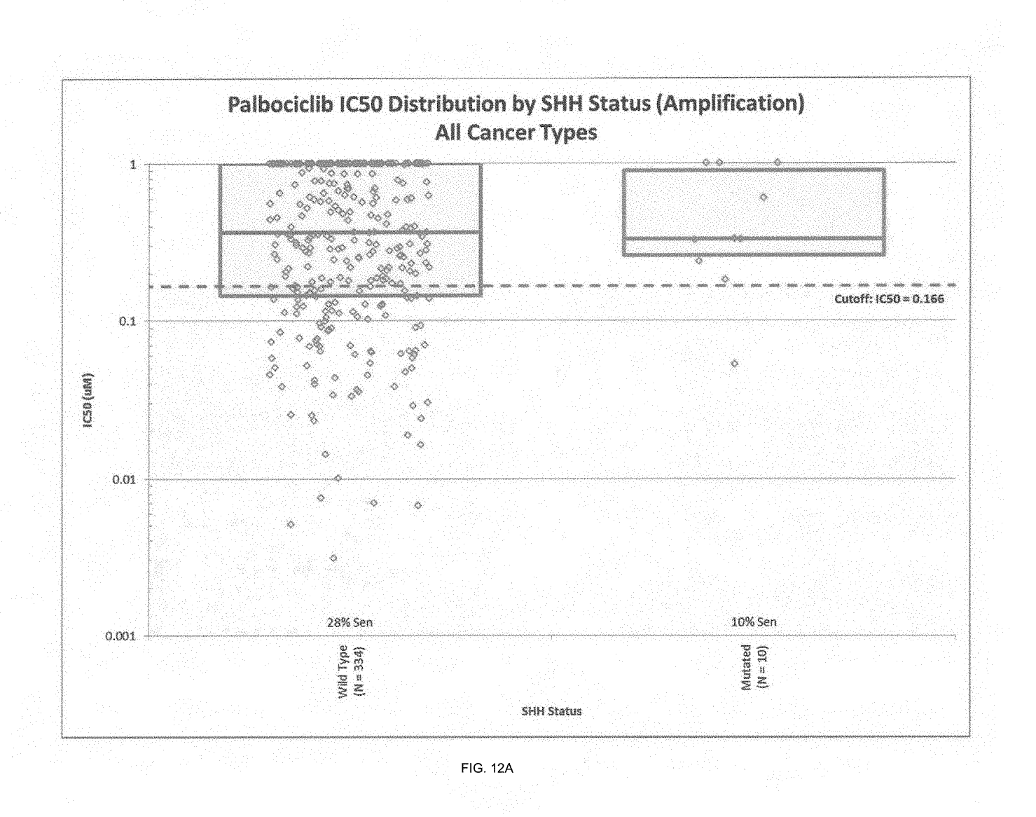

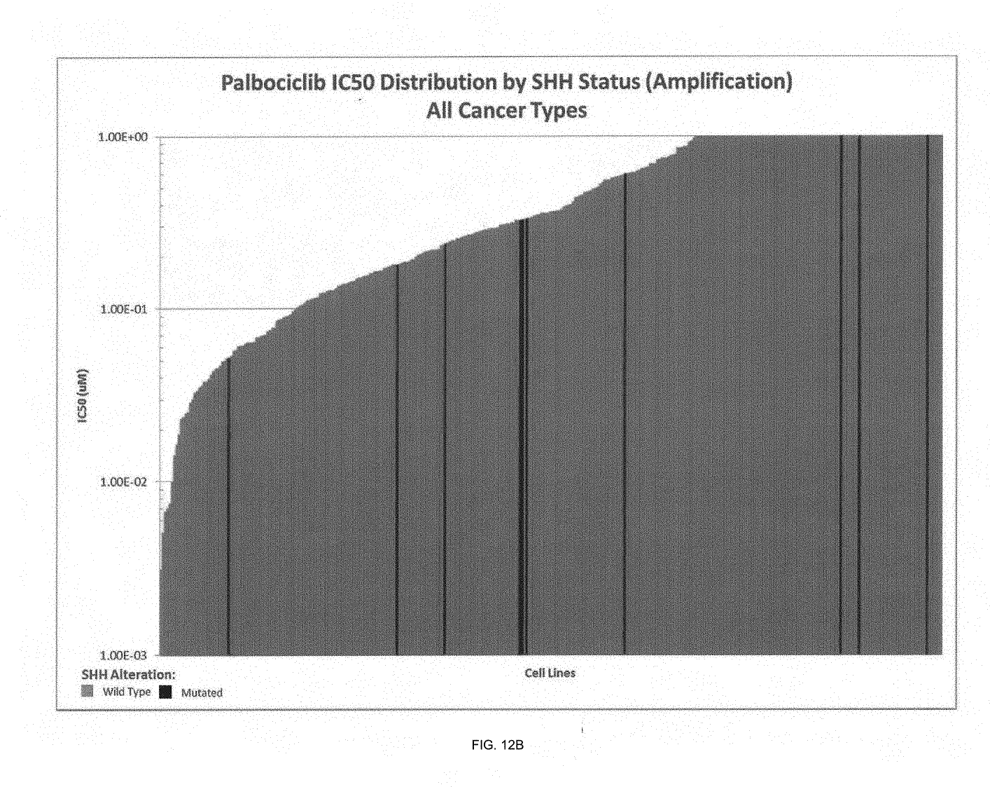

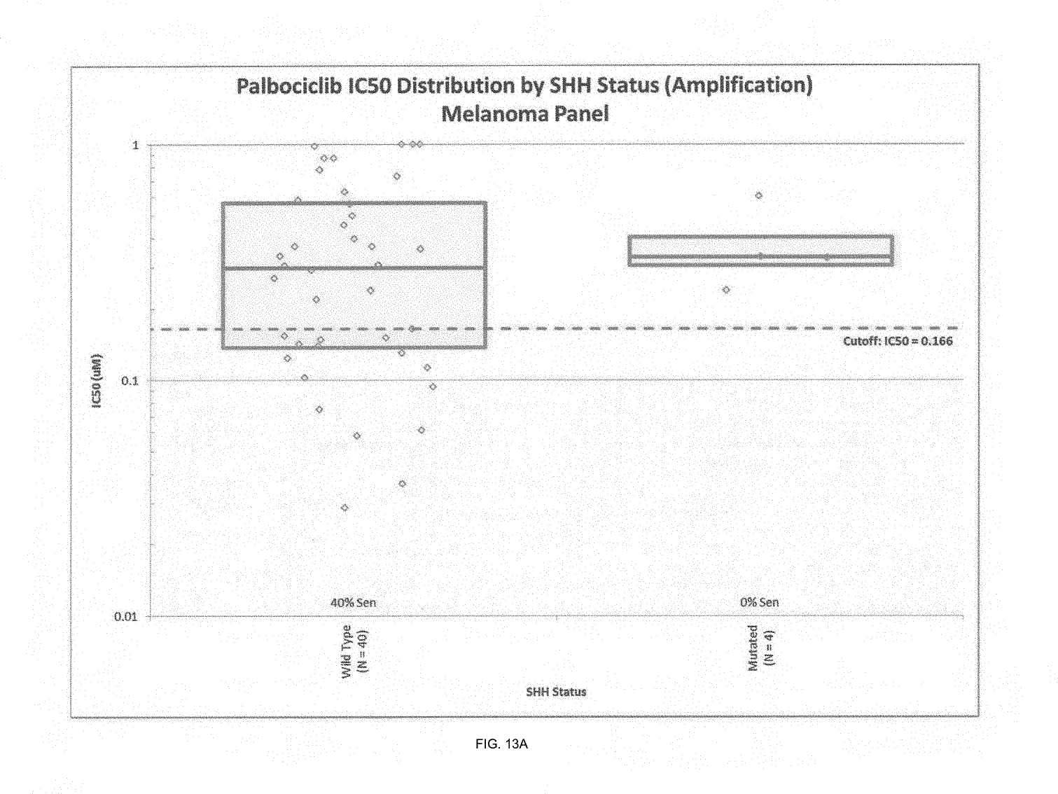

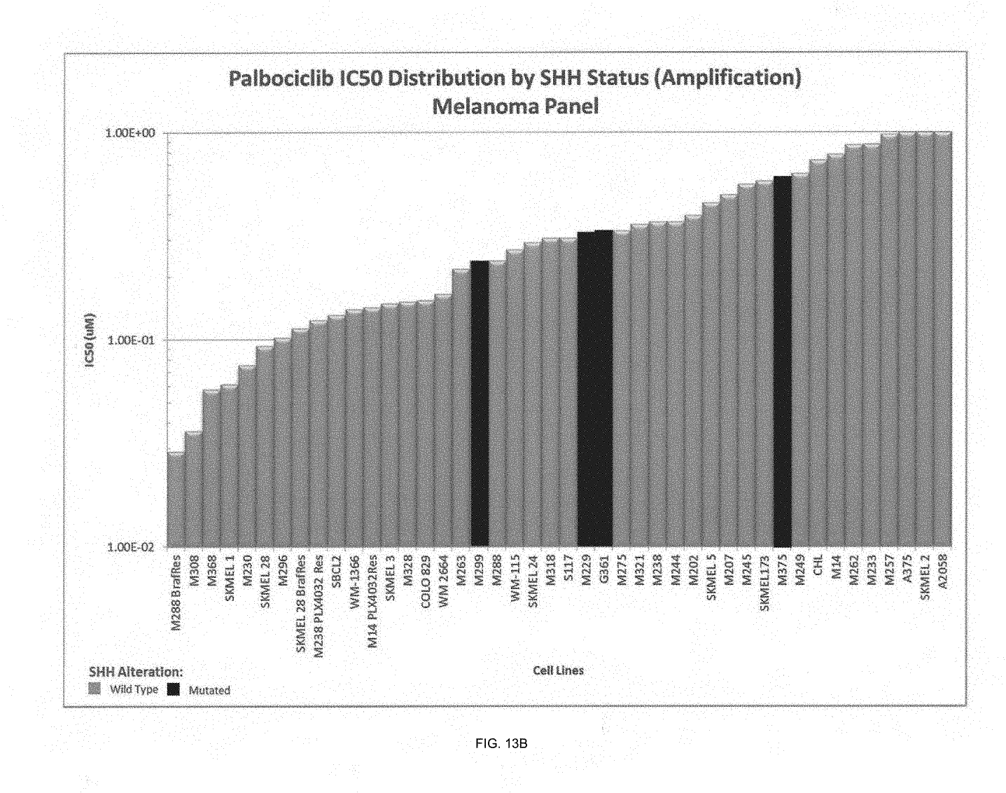

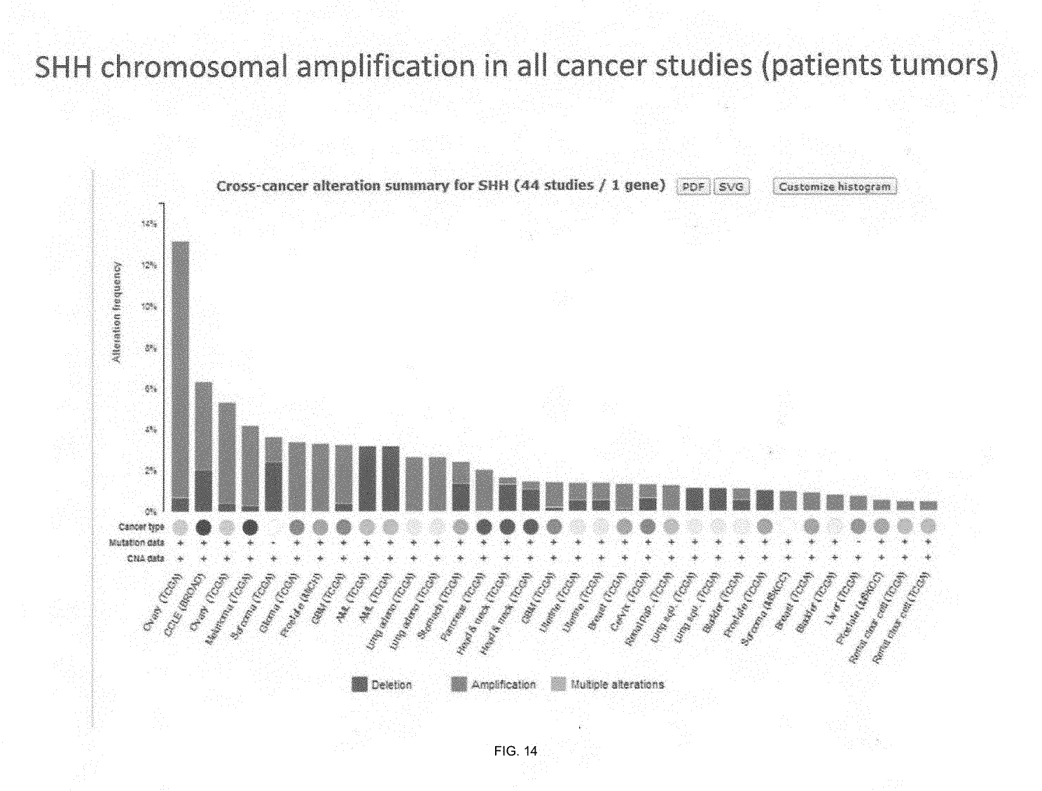

While the disclosure has been described with reference to the above and to Attachments 1 and 2, which are incorporated herein by reference (see Examples 1-3 and FIGS. 1-16), it will be understood that it is capable of further modifications. This application is intended to cover any variations, uses, or adaptations of the disclosure following, in general, the disclosed principles and including such departures from the disclosure as come within known or customary practice within the art to which the disclosure pertains and as may be applied to the essential features hereinbefore set forth. Accordingly, the invention is limited only by the following claims.

### EXAMPLES

### Example 1

### Identification of Genomic Predictors of Response to the CDK4/6 Inhibitor Palbociclib Using the UCLATORL Panel of Human Cancer Cell Lines

Palbociclib is a highly specific small molecule inhibitor of cyclin-dependent kinases 4 and 6 currently in clinical development by Pfizer. It is designed to inhibit the cell cycle at the G1/S transition via preventing the phosphorylation of Rb by the Cyclin D/CDK4/6 enzyme complex. Our lab previously identified the estrogen receptor positive subpopulation of breast cancer patients as distinctively likely to benefit from treatment with palbociclib. This observation spurred the initiation of a Phase II clinical trial in this patient population where remarkable efficacy was observed. Given this translational success, we wished to investigate the molecular determinants of response to palbociclib in several additional cancer types.

To this end, we assayed the in vitro sensitivity to palbociclib across a panel of 416 cancer cell lines derived from 12 distinct cancer types. We observed highly differential response to treatment both within and between cancer types. IC50s (the concentration of palbociclib required to inhibit fifty percent of population doublings) ranged from the low nanomolar range to above the highest dose tested (1(JM). This response distribution was much broader than was observed in similar screens performed by the Broad and Sanger institutes, where the vast majority of cell lines assayed were listed as having IC50s above 1 uM. Our ability to identify a higher proportion of palbociclib-sensitive cell lines can likely be credited to methodological innovations aimed at optimizing our screening protocol for the detection of longer-term cytostatic effects, as opposed to shorter-term cytotoxic effects of treatment. The generation of this highly differential response dataset allowed for a unique opportunity to explore the possible genetic mechanisms underlying differential sensitivity to treatment with palbociclib in vitro.

The other dataset interrogated for genotype-response associations was a copy number alteration dataset derived from comparative genomic hybridization arrays. This dataset was organized by gene and also restricted by various criteria to enrich for amplifications or deletions of genes likely to be causally involved in carcinogenesis. From this dataset we identified three chromosomal regions (17q12-21, 11q13, 1q32) where amplification was associated with sensitivity to palbociclib. Two amplified regions (19q13, 8q13) were found to be associated with resistance. Homozygous deletions of the 13q14 chromosomal region were found to be strongly associated with resistance to palbociclib.

Following these extensive post-hoc analyses, we pruned our original set of fourteen biomarkers down to the eight most likely to play a causal role in determining sensitivity or resistance to treatment with palbociclib. The final set of candidate sensitivity biomarkers included: loss-of-function point mutations in CDH1, loss-of-function point mutations in TOPBP1, chromosomal amplification of 17q12-21 (ERBB2) and chromosomal amplification of 11q13 (CCND1). The final set of candidate resistance biomarkers included: activating point mutations in SMO, chromosomal amplification of CCNE1, loss-of-function point mutations in RB1 and chromosomal deletion of 13q14 (RB1) (FIG. 1 to FIG. 11)

This final set of eight candidate biomarkers was analyzed by strata representing each of the 12 cancer types in our cell line panel. The four candidate sensitivity biomarkers were sufficiently frequent and associated with sensitivity in 6 of the 12 cancer types in our panel. These were the breast, colon, head/neck, lung, ovarian and upper gastrointestinal strata. The four candidate resistance biomarkers were sufficiently frequent and associated with resistance in 6 of the 12 cancer types in our panel. These were the breast, colon, kidney, lung, ovarian and upper gastrointestinal strata.

### Example 2

### Identification of Markers of Sensitivity and Resistance to Palbociclib (PD0332991) in Melanoma

Melanoma, a malignancy originating in pigment-producing melanocytes, is generally resistant to conventional treatments such as radiotherapy or chemotherapy. Molecular targeted therapeutics against BRAF and MEK have shown marked efficacy for patients with metastatic melanoma. However these targeted therapies have a limited duration of response. Palbociclib is a highly selective inhibitor of cyclin dependent kinases 4 and 6 (CDK4/6) that has been shown to inhibit growth of malignant cell lines in vitro and in vivo by preventing the phosphorylation of Rb and stopping the progression G1/S of cell cycle. The Cyclin D-CDK4/6-Rb axis has been shown to be dysregulated in 85-90% of melanomas and may represent a new therapeutic target. Our main goal in this study was to evaluate the therapeutic potential of palbociclib in a large panel of human melanoma cell lines to identify potential biomarkers of sensitivity and resistance.

Material and Methods: We evaluated the response palbociclib treatment in 48 melanoma cell lines. A comprehensive biomarker screen was performed using mutation and gene expression (Agilent) data. The mechanism of action of the drug, phospho-Rb and senescence, was investigated by western blot (WB) analysis. Effects on the cell cycle and apoptosis were study by flow cytometry.

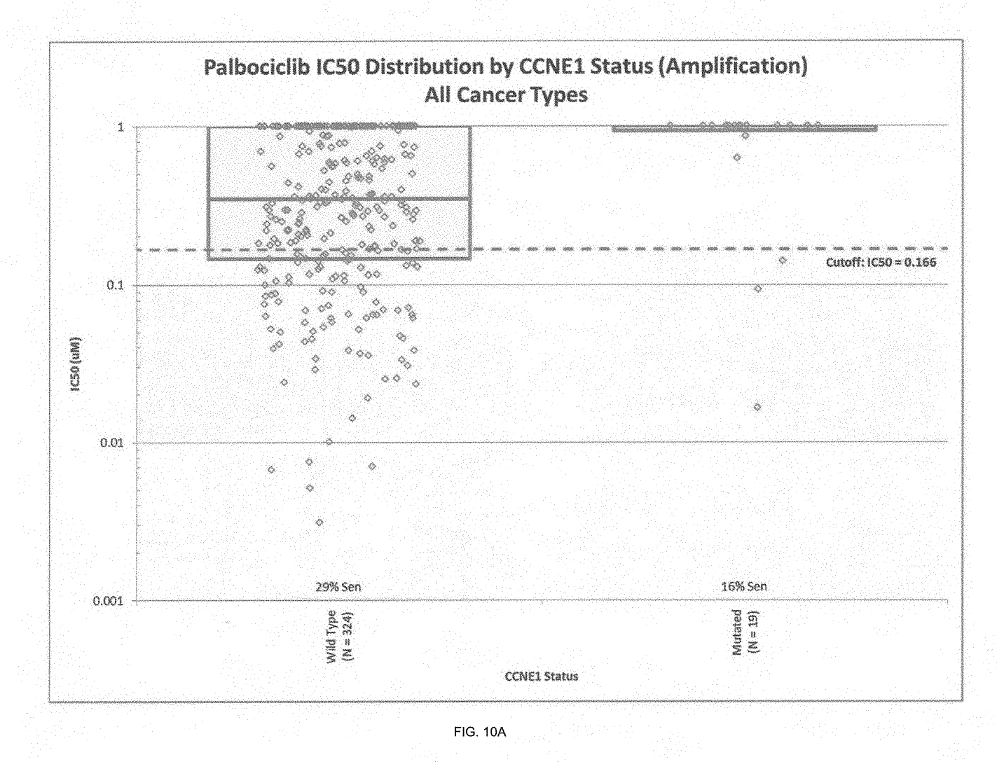

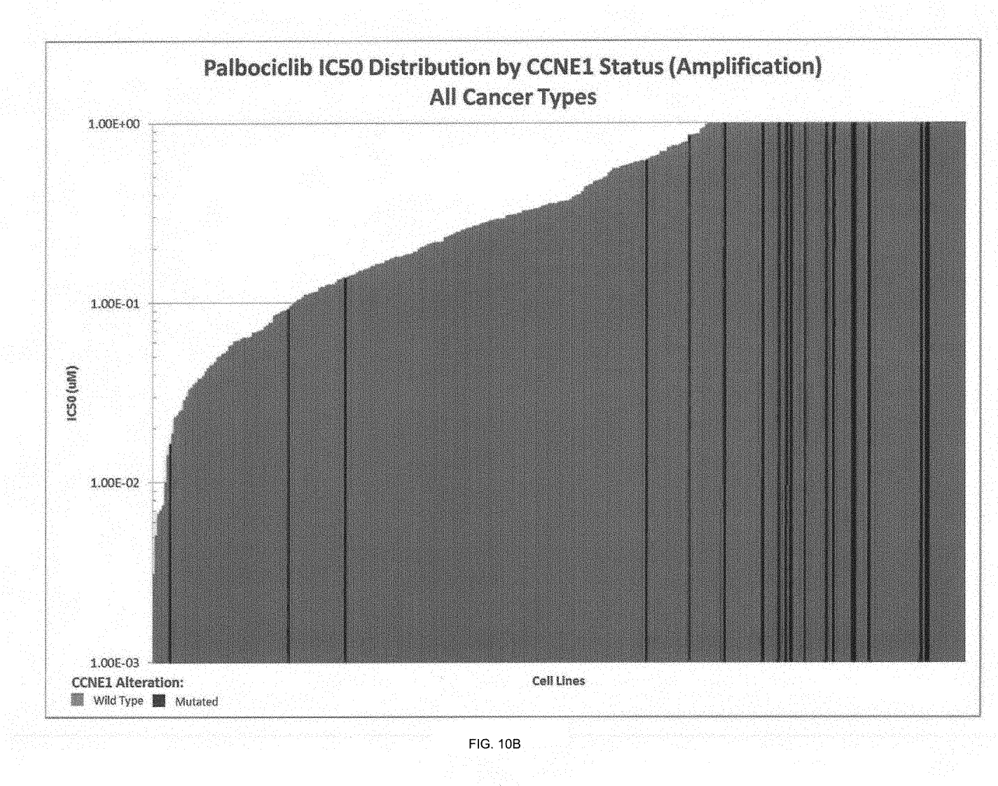

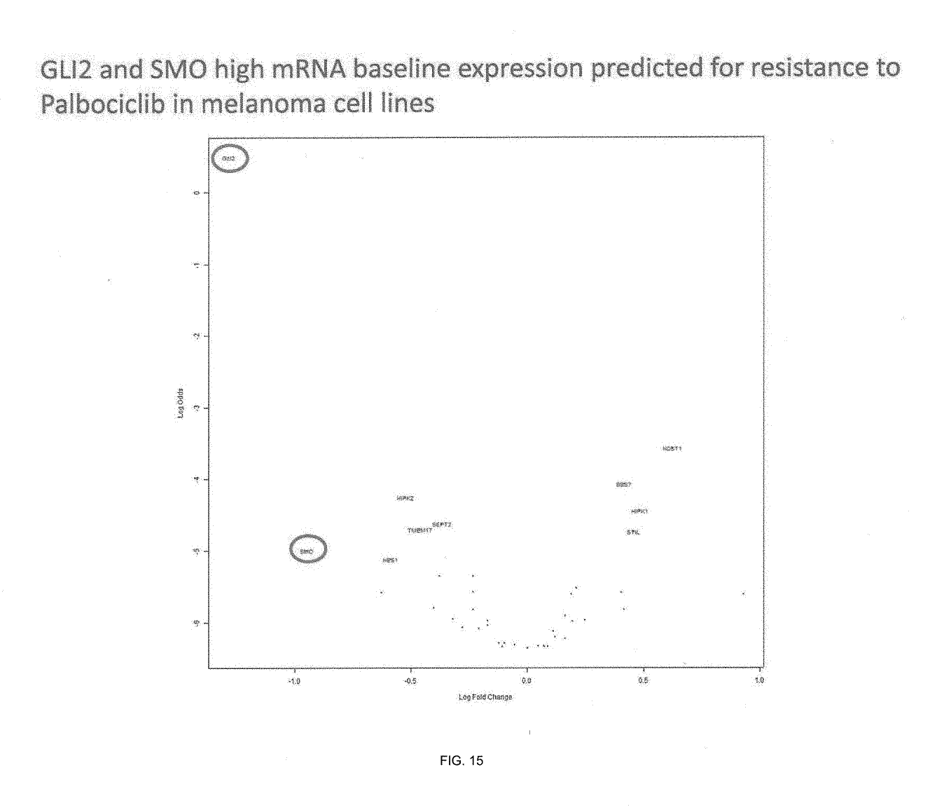

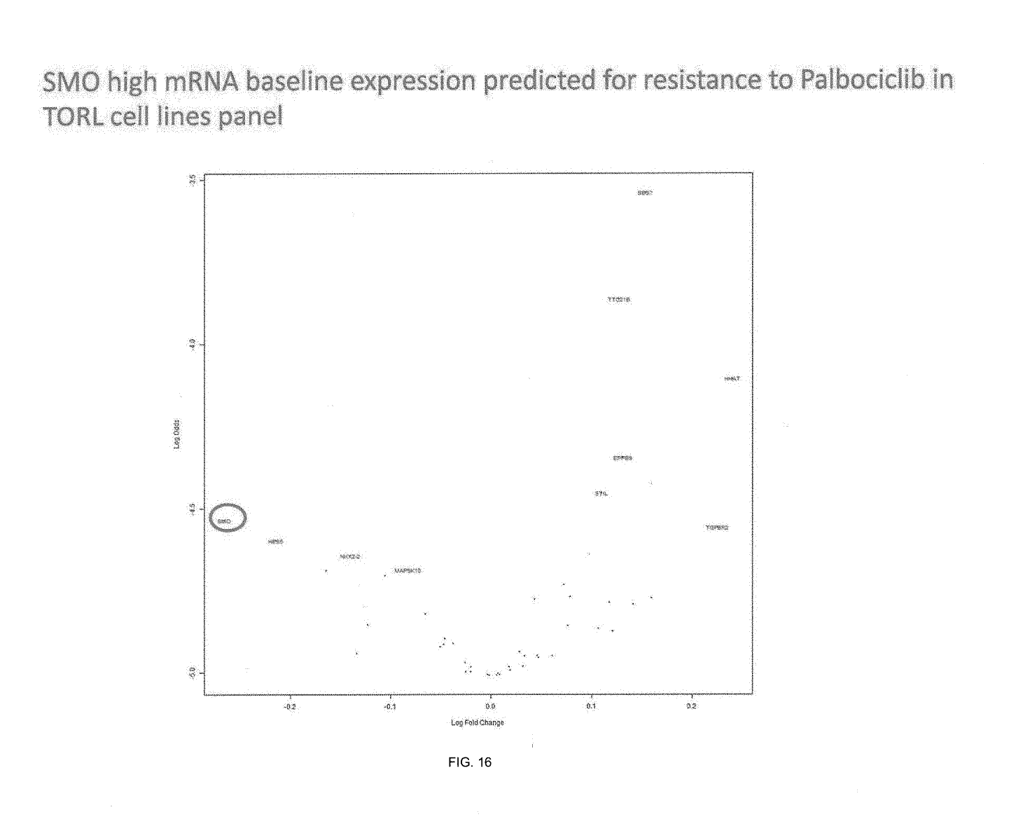

Results: A marked differential response to palbociclib was observed across the melanoma cell lines with IC50s ranging from 28 nM to over 1 μM. A subset (14/48) of cell lines were classified as sensitive at clinically achievable concentrations (IC50<150 nM). BRAF and NRAS activating mutation did not correlate with sensitivity in vitro. Increased expression of genes correlating with cyclin D-CDK4/6 activation strongly predicted for palbociclib sensitivity. We also found that genomic and expression markers of activation of the hedgehog/smoothened (SHH) pathway strongly predicted for in vitro resistance to palbociclib (FIGS. 12-14). These markers included chromosomal amplification of SHH, and high baseline expression of SMO and GLI2 (FIGS. 15-16). Protein analysis by Western blot showed a dramatic decrease in phospho-Rb protein only in sensitive cell lines and also it caused a decrease in FOXM1 protein, indicating palbociclib may partially act by inducing senescence in treated cells. Flow cytometry analysis confirmed that palbociclib induces a strong G1/S arrest, but not significant apoptosis.

Conclusion: Nearly 30% of the melanoma cell lines evaluated were found to be sensitive to palbociclib in vitro. Response to palbociclib did not correlate with BRAF and NRAS mutational status. A gene expression signature for CDK 4/6 activation successfully identified those cell lines most sensitive to palbociclib. We also identified markers of hedgehog/smoothened/GLI activation that strongly predicted for resistance to palbociclib. These data support the development of CDK 4/6 inhibitors in melanoma and provide a hypothesis for patient enrichment in clinical trials.

Additional studies were undertaken on sensitive and resistant melanoma cell lines. Cell lines representative of the spectrum of sensitivities were cultured in the presence of 100 nM palbociclib for 72 hours and stained with DAPI for cell cycle analysis. Most of the cell lines showed a G0-G1 arrest after palbociclib treatment. This arrest was more prominent on the sensitive cells lines (FIG. 17). Effects of palbociclib treatment on FoxM1, RB, CDK4 and CDK6 protein expression. M14 (sensitive) and A375 (resistant) cells were exposed for 30 min, 24 and 48 hrs to media control or 100 nM palbociclib. Western blot analysis showed a decrease on phospho-Rb protein in sensitive cells. Decrease of FoxM1 is associated with cellular senescence, palbociclib treatment caused a decrease in this protein only in sensitive cells.

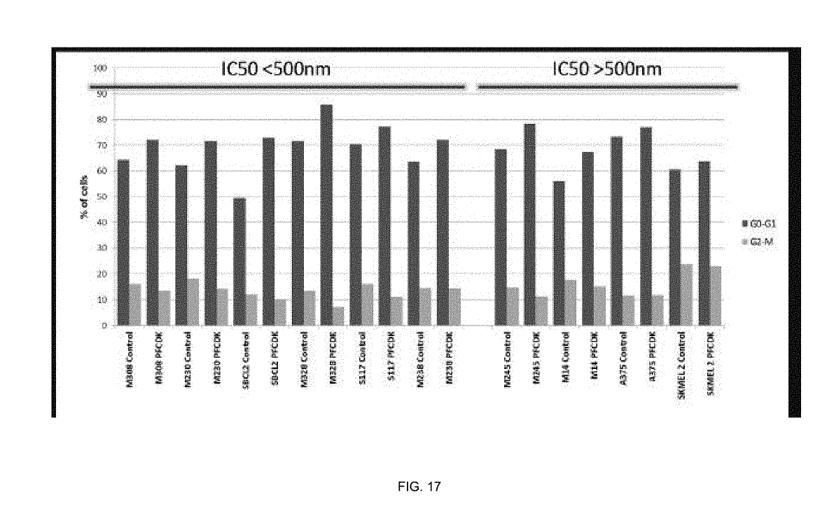

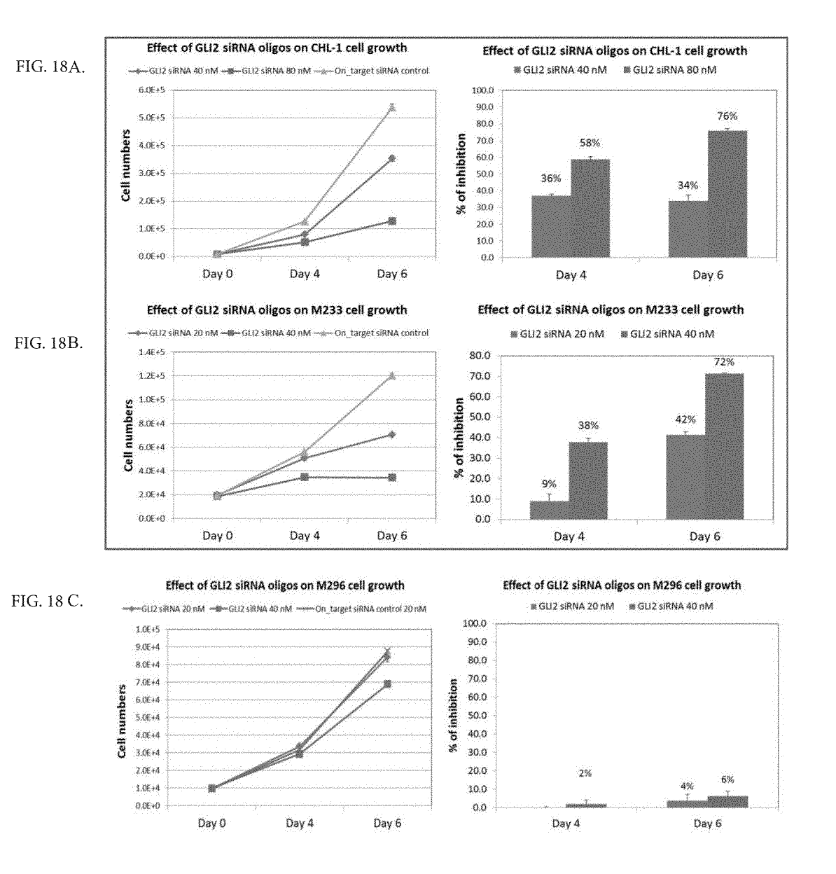

To explore the functional relevance of GLI2 expression in palbociclib resistant cell lines CHL-1 (FIG. 18A) and M233 (FIG. 18B), knockdown studies were performed using siRNA. Palbociclib sensitive cells M296 (FIG. 18C) was used as a control. Total cell number (left panels) and percent inhibition were assessed (right panels). Reduction of GLI2, assessed by Western Blot, significantly reduced the cell growth proliferation, confirming the SMO/GLI2 cell cycle dependency, instead of CDK-4/6-Cyclin D pathway.

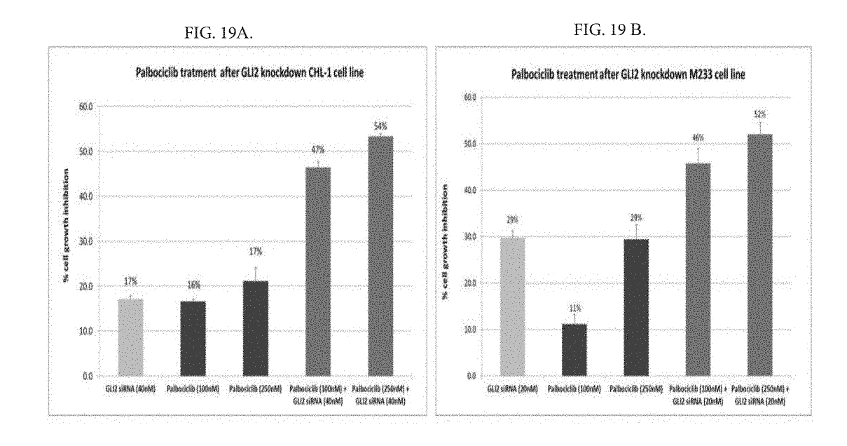

Effects of palbociclib treatment on resistance cells in combination with GLI2 siRNA knockdown on cell lines expressing high GLI2 protein levels was determined. GLI2 was knocked down and palbociclib was added at two different doses 100 nM and 250 nM. Assessment on the cell growth proliferation after 6 days of treatment showed a potentiation in the inhibition of the cell growth proliferation (FIG. 19A and FIG. 19B).

These results indicate that Smoothened and CDK-4/6 inhibitors have the potential to have a synergistic effect in the clinic. Exemplary inhibitors of Smoothened include GDC-0449, cyclopamine, itraconazole, LDE225, IPI-926 and vismodegib (Erivedge).

### Example 3

### Expression of p16 in Colon Cancer and Cyclin D1 in Gastric Cancer Predicts Response to CDK4/6 Inhibition In Vitro

Cyclin-dependent kinases (CDKs) play a significant role in regulating cell cycle progression through association with cyclins. CDK4 and CDK6 interact with cyclin D1 to mediate hyperphosphorylation of retinoblastoma (Rb) during early G1 phase. Palbociclib is a highly selective inhibitor of CDK4 and CDK6 which functions by blocking pRb phosphorylation resulting in Gi arrest in sensitive cell lines. We evaluated the effect of palbociclib in gastric and colon cancer cell lines to explore potential biomarkers of response and to guide patient selection in colon and gastric cancer.

Methods: Panels of 17 gastric and 27 colon cancer cell lines were exposed in vitro to palbociclib over various concentrations to generate dose response curves. Analysis of variance (ANOVA) was used to identify differentially expressed genes between sensitive and resistant cell lines. Genes identified by ANOVA and effects of palbociclib on pRB were analyzed via western blot.

Results: Palbociclib was found to have potent anti-proliferative activity in the low nanomolar range (<150 nM) for 14 of the 44 gastric and colon cancer cell lines tested. In gastric cancer, cyclin D1-amplified cells expressed greater sensitivity to the compound when compared to cyclin D1-negative cells. HER2 amplified cell lines were also statistically more sensitive than HER2 negative cells. Furthermore, cyclin E emerged as a biomarker for resistance to the compound in gastric cancers. Contrary to observations made in other cancers, expression levels of p16 (CDK4 inhibitor) and p21 (CDK2 inhibitor) in colon cancer indicated that p16 loss and p21 gain predict for resistance rather than sensitivity to CDK4 and CDK6 inhibition.

### CONCLUSIONS

Palbociclib demonstrates anti-proliferative activity in several gastric and colon cancer cell lines. Molecular markers found to predict for sensitivity to this agent enhance patient selection for future clinical studies of palbociclib.

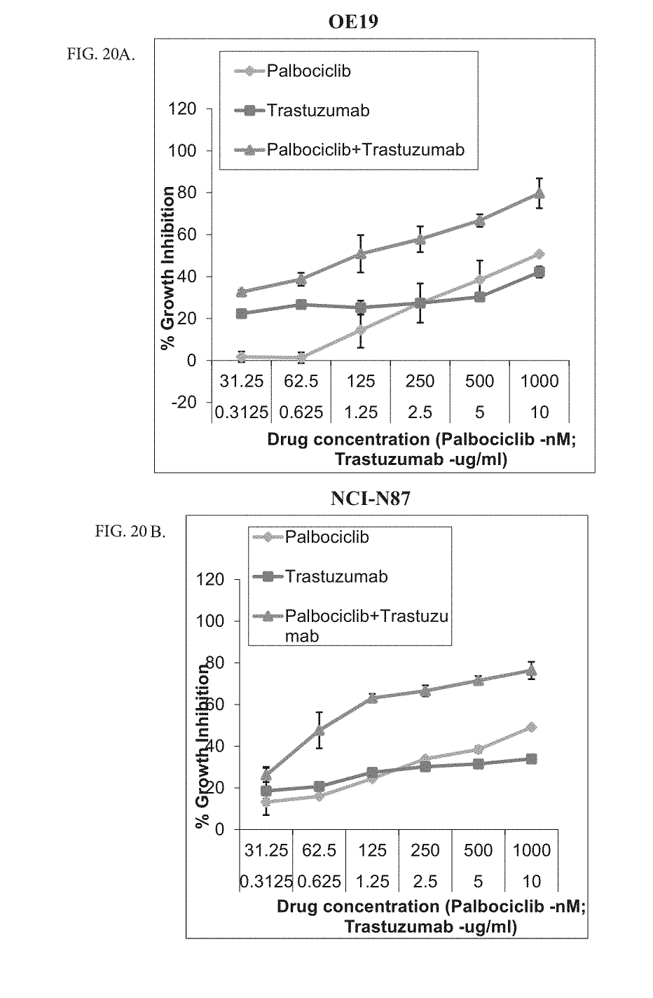

Additional studies were undertaken to determine the effects of palbociclib in gastric and colon cancer cell lines. To evaluate the combination of palbociclib and trastuzumab, two HER2-amplified gastric cancer cell lines (OE19 and NCI-N87) were treated with various doses. When these two agents were combined, significant synergy was observed in both HER2-amplified cell lines (FIG. 20A and FIG. 20B). Mean combination indices (CI) were obtained ranging from 0.14±0.06 in NCI-N87 to 0.69±0.04 in OE19.

Analysis of variance (ANOVA) identified differential gene expression levels of both p16 (CDK4 inhibitor) and p21 (CDK2 inhibitor) in colon and gastric cancer cells. Contrary to observations made in other cancers, loss of p16 (CDKN2A) in colon and gastric cancer as well as gain of p21(CDKN1A) in colon lines predict for resistance rather than sensitivity to CDK4 and CDK6 inhibition in vitro.

The effects of palbociclib on select markers downstream of CDK4 and CDK6 proteins was examined in both sensitive (SW480 and MKN74) and resistant (HCT8 and MKN1) gastric and colon cell lines. There was time-dependent inhibition of Rb phosphorylation and decreased expression of total Rb after treatment with palbociclib which was more pronounced in the sensitive lines (SW480 and MKN74).

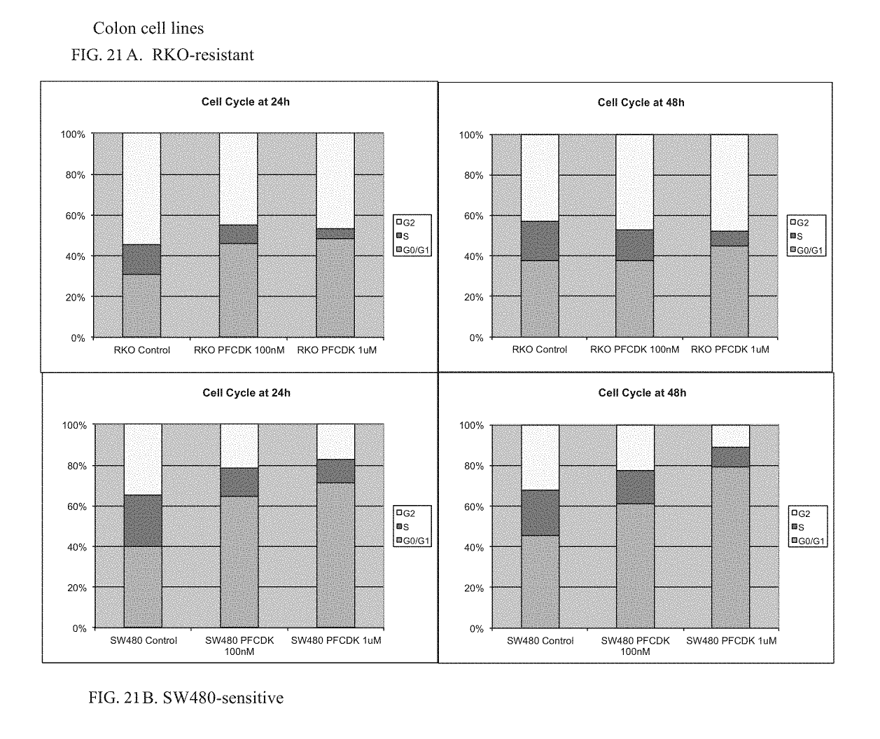

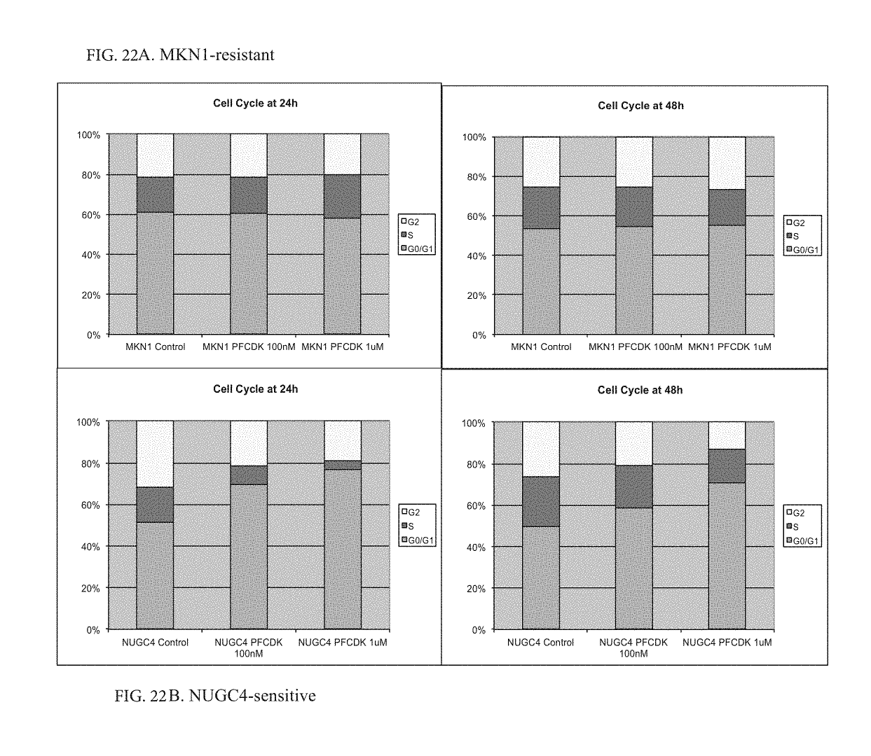

Cell cycle analysis was used to compare the sensitive and resistant gastric and colon cancer cell lines. In the cells that were sensitive to the anti-proliferative effects of palbociclib (SW480 and NUGC4), palbociclib induced a potent G1/G0 arrest at both 24-hour and 48-hour time points. In contrast, in the resistant lines cell lines (RKO and MKN1), there was a minimal effect (FIG. 21A-FIG. 21B and FIG. 22A-FIG. 22B).

These results how that similar to observations made in breast cancer, gastric and colon cancer Cyclin D-amplified cells typically expressed greater sensitivity to palbociclib. In gastric cancer cell lines, those that were HER2-amplified were particularly sensitive to palbociclib and had a synergistic effect with trastuzumab. ANOVA analysis of baseline gene expression identified p16 loss (in both colon and gastric lines) and p21 gain (in colon) as potential predictors for resistance to palbociclib. Clinical trials combining CDK 4/6 inhibitors with trastuzumab in HER2 amplified cancers are ongoing. Molecular markers found to predict for sensitivity to this agent enhance patient selection for future clinical studies of palbociclib

1-37. (canceled)

38. A method of testing a subject having cancer, the method comprising:
(a) obtaining a tumor sample from the subject;
(b) assaying for the presence of at least one biomarker selected from the group consisting of:
(i)) a chromosomal deletion of RB1,
(ii) a chromosomal amplification of 19q12 (CCNE1),
(iii) loss of p16,
(iv) an activating point mutation of the smoothened (SMO) gene,
(v) an activating point mutation of the RET proto-oncogene,
(vi) a loss-of-function point mutation of FBXW7,
(vii) a loss-of-function point mutation of retinoblastoma (RB1),
(viii) a chromosomal amplification of sonic hedgehog (SHH),
(ix) high baseline GLI2 mRNA expression,
(x) high baseline SMO mRNA expression, and
(xi) p21 gain,
wherein the presence of the at least one biomarker indicates that the subject is unlikely to respond to a treatment with at least one CDK4 inhibitor-, one CDK6 inhibitor-, or one cyclin D inhibitor, or combinations thereof.

39. The method of claim 38, wherein the testing comprises identifying the subject as having a tumor that is CDK4 inhibitor-, CDK6 inhibitor-, or cyclin D inhibitor-resistant.

40. The method of claim 38, wherein the at least one biomarker is amplified in a chromosomal region selected from the group consisting of 19q13 and 8q13.

41. The method of claim 38, wherein the at least one biomarker is homozygously deleted in a 13q14 chromosomal region.

42. The method of claim 38, wherein at least two biomarkers are assayed.

43. The method of claim 38, wherein at least three biomarkers are assayed.

44. The method of claim 38, wherein the at least one biomarker is a chromosomal deletion of RB1.

45. The method of claim 38, wherein the at least one biomarker is a chromosomal amplification of 19q12 (CCNE1).

46. The method of claim 38, wherein assaying comprises a single nucleotide polymorphism (SNP) array, comparative genomic hybridization (CGH), southern blot analysis, fluorescent in situ hybridization (FISH), or chromogenic in situ hybridization (CISH).

47. The method of claim 38, wherein assaying comprises ELISAs, Multiplex (MSD), reverse phase protein analysis-RPPA, immunohistochemical staining analyses, or antibody-based methodology.

48. The method of claim 38, wherein the chromosomal amplification is determined by comparison to a genome of a normal cell.

49. The method of claim 38, wherein the tumor is selected from the group consisting of a melanoma, a breast cancer, a bladder cancer, an ovarian cancer, a lung cancer, a head and neck cancer, an upper gastrointestinal cancer, a kidney cancer, and a colon cancer.

50. A method of testing a subject having cancer, the method comprising:
(a) obtaining a tumor sample from the subject;
(b) assaying for the presence of at least one biomarker selected from the group consisting of:
(i) copy number amplification of RB1,
(ii) chromosomal amplification of 17q12-21 (ERBB2),
(iii) chromosomal amplification of 11q13 (CCND1),
(iv) chromosomal amplification of 1q (MDM4),
(v) high p16 protein,
(vi) a loss-of-function point mutation in CDH1,
(vii) cyclin D1 amplification,
(viii) HER2 amplification,
(ix) loss-of-function point mutations in TOPBP1,
(x) decrease in phospho-Rb, and
(xi) decrease in FOXM1 protein,
wherein the presence of the at least one biomarker indicates that the subject is likely to respond to a treatment with at least one CDK4 inhibitor-, CDK6 inhibitor-, cyclin D inhibitor, or combinations thereof.

51. The method of claim 50, wherein the method identifies the subject as having a tumor that is CDK4 inhibitor-, CDK6 inhibitor-, or cyclin D inhibitor-sensitive.

52. The method of claim 50, wherein the at least one biomarker is amplified in a chromosomal region selected from the group consisting of 17q12-21, 11q13, and 1q32.

53. The method of claim 50, wherein at least two biomarkers are assayed.

54. The method of claim 50, wherein the tumor is colon cancer, and the at least one biomarker comprise copy number amplification of RB1.

55. The method of claim 50, wherein assaying comprises a single nucleotide polymorphism (SNP) array, comparative genomic hybridization (CGH), southern blot analysis, fluorescent in situ hybridization (FISH), or chromogenic in situ hybridization (CISH).

56. The method of claim 50, wherein assaying comprises ELISAs, Multiplex (MSD), reverse phase protein analysis-RPPA, western blot analysis, immunohistochemical staining analyses, or antibody-based methodology.

57. The method of claim 50, wherein the chromosomal amplification is determined by comparison to a genome of a normal cell.

58. The method of claim 50, wherein the tumor is selected from the group consisting of a melanoma, a breast cancer, an ovarian cancer, a lung cancer, a head and neck cancer, an upper gastrointestinal cancer, a kidney cancer, and a colon cancer.
